(19) United States
(12) Patent Application Publication
(10) Pub. No.: US 2007/0105892 A1
(43) Pub. Date: May 10, 2007

(54) Amidino compounds as cysteine protease inhibitors

(75) Inventors: Michael Graupe, Pacifica, CA (US); Agnes J. Lau, San Francisco, CA (US); Jiayao Li, Foster City, CA (US); John O. Link, San Francisco, CA (US); Craig J. Mossman, Campbell, CA (US); Soon H. Woo, Palo Alto, CA (US); Sheila M. Zipfel, Mountain View, CA (US)

Correspondence Address: BERLEX BIOSCIENCES;PATENT DEPARTMENT, 2600 HILLTOP DRIVE, P.O. BOX 4099, RICHMOND, CA, 94804-0099, US

(73) Assignee: AXYS Pharmaceuticals, Inc., South San Francisco, CA (US)

(21) Appl. No.: 10/583,629

(22) PCT Filed: Dec. 22, 2004
(86) PCT No.: PCT/US04/43451

§ 371 Date: Jan. 10, 2007

Related U.S. Application Data
(60) Provisional application No. 60/532,243, filed on Dec. 23, 2003.

(51) Int. Cl. A61K 31/4745; A61K 31/4245; C07D 491/02; C07D 271/10

(52) U.S. Cl. 514/302; 514/364; 514/375; 546/114; 546/269.1; 548/143

(57) ABSTRACT

The present invention is directed to compounds that are inhibitors of cysteine proteases, in particular, cathepsins B, K, L, F, and S and are therefore useful in treating diseases mediated by these proteases. The present invention is directed to pharmaceutical compositions comprising these compounds and processes for preparing them.

### FIELD OF THE INVENTION

The present invention is directed to compounds that are inhibitors of cysteine proteases, in particular, cathepsins B, K, L, F, and S and are therefore useful in treating diseases mediated by these proteases. The present invention is also directed to pharmaceutical compositions comprising these compounds and processes for preparing them.

### STATE OF THE ART

Cysteine proteases represent a class of peptidases characterized by the presence of a cysteine residue in the catalytic site of the enzyme. Cysteine proteases are associated with the normal degradation and processing of proteins. The aberrant activity of cysteine proteases, e.g., as a result of increased expression or enhanced activation, however, may have pathological consequences. In this regard, certain cysteine proteases are associated with a number of disease states, including arthritis, muscular dystrophy, inflammation, tumor invasion, glomerulonephritis, malaria, periodontal disease, metachromatic leukodystrophy, and others. For example, increased cathepsin B levels and redistribution of the enzyme are found in tumorsthus, suggesting a role for the enzyme in tumor invasion and metastasis. In addition, aberrant cathepsin B activity is implicated in such disease states as rheumatoid arthritis, osteoarthritis, pneumocystis carinii, acute pancreatitis, inflammatory airway disease and bone and joint disorders.

The prominent expression of cathepsin K in osteoclasts and osteoclast-related multinucleated cells and its high collagenolytic activity suggest that the enzyme is involved in ososteoclast-mediated bone resorption and hence, in bone abnormalities such as occurs in osteoporosis. In addition, cathepsin K expression in the lung and its elastinolytic activity suggest that the enzyme plays a role in pulmonary disorders as well.

Cathepsin L is implicated in normal lysosomal proteolysis as well as several disease states, including, but not limited to, metastasis of melanomas. Cathepsin S is implicated in Alzheimer's disease and certain autoimmune disorders, including, but not limited to juvenile onset diabetes, multiple sclerosis, pemphigus vulgaris, Graves' disease, myasthenia gravis, systemic lupus erythemotasus, rheumatoid arthritis and Hashimoto's thyroiditis. In addition, cathepsin S is implicated in allergic disorders including, but not limited to asthma and allogeneic immune reponses including, but not limited to, rejection of organ transplants or tissue grafts.

Another cysteine protease, Cathepsin F, has been found in macrophages and is involved in antigen processing. It is believed that Cathepsin F in stimulated lung macrophages and possibly other antigen presenting cells could play a role in airway inflammation (see G. P. Shi et al, J. Exp. Med. 2000, 191, 1177)

In view of the number of diseases wherein it is recognized that an increase in cysteine protease activity contributes to the pathology and/or symptomatology of the disease, molecules which inhibit the activity of this class of enzymes, in particular molecules which inhibitor cathepsins B, K, L, F, and/or S, are therefore useful as therapeutic agents.

### SUMMARY OF THE INVENTION

In one aspect, this invention is directed to a compound of Formula (I): 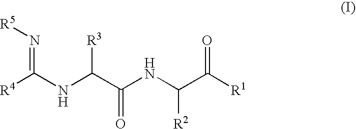
wherein:

R¹ is benzoxazol-2-yl, oxazolo-[4.5-b]-pyridin-2-yl, 2-ethyl-[1.3.4]-oxadiazol-5-yl, 2-phenyl-[1.3.4]-oxadiazol-5-yl, 3-phenyl-[1.2.4]-oxadiazol-5-yl, 3-thien-3-yl-[1.2.4]-oxadiazol-5-yl, 3-pyridin-3-yl-[1.2.4]-oxadiazol-5-yl, 3-ethyl-[1.2.4]-oxadiazol-5-yl, 5-ethyl-[1.2.4]-oxadiazol-3-yl, or 2-methoxymethyl-[1.3.4]-oxadiazol-5-yl; and

R² is ethyl or n-propyl;

R³ is cylohexylmethyl, 1-methylcyclohexylmethyl, cyclopentylmethyl, 1-methylcyclopentylmethyl, cyclopropylmethylsulfinylmethyl, cyclopropylmethylsulfonylmethyl, 2-phenylsulfanylethyl, 2-phenylsulfonylethyl, pyridin-2-ylmethylsulfonylmethyl, benzylsulfinylmethyl, benzylsulfonylmethyl, 2-(difluoromethoxy)-benzylsulfonylmethyl, or 2-chlorobenzyl;

R⁴ is methyl, phenyl, 4-fluorophenyl, isopropylamine, cyclopentylamine, tetrahydropyran-4-yl, morpholin-4-yl, or pyrrolidin-1-yl;

R⁵ is methylsulfonyl, 2,2,2-trifluoroethyl, ethoxycarbonyl, or pyridin-3-ylsulfonyl; or

R⁴ and R⁵ together with the atoms to which they are attached form 1,1-dioxo-benzo[d]isothiazol-3-yl or 1,1-dioxo-1,4-dihydro-λ⁶-benzo[1,2,4]thiadiazin-3-yl; or a pharmaceutically acceptable salt thereof.

Preferably, R³ is 1-methylcyclopentylmethyl.

Preferably, a compound selected from the group consisting of: 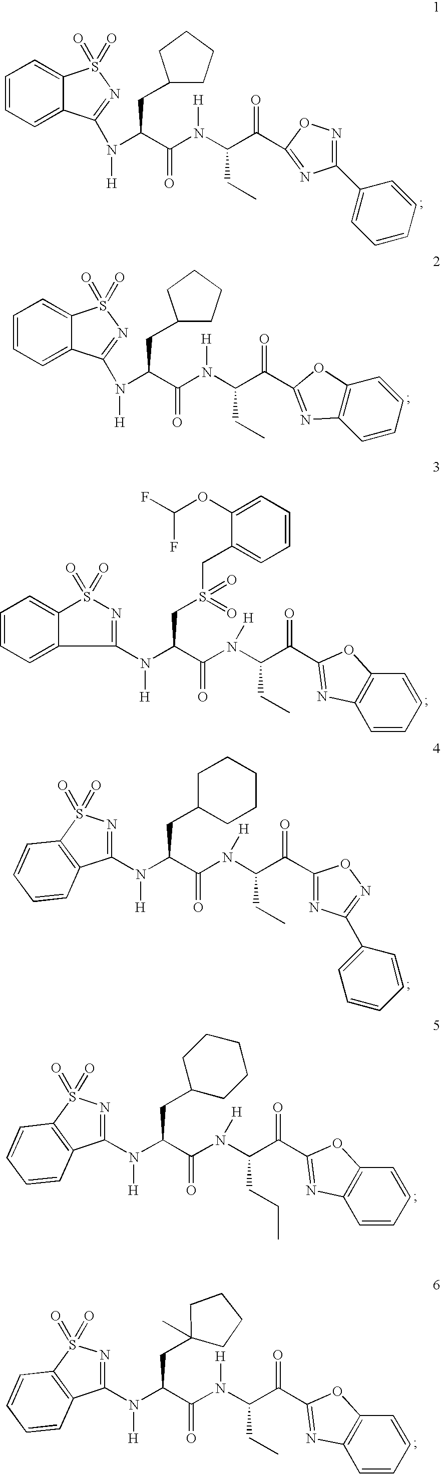 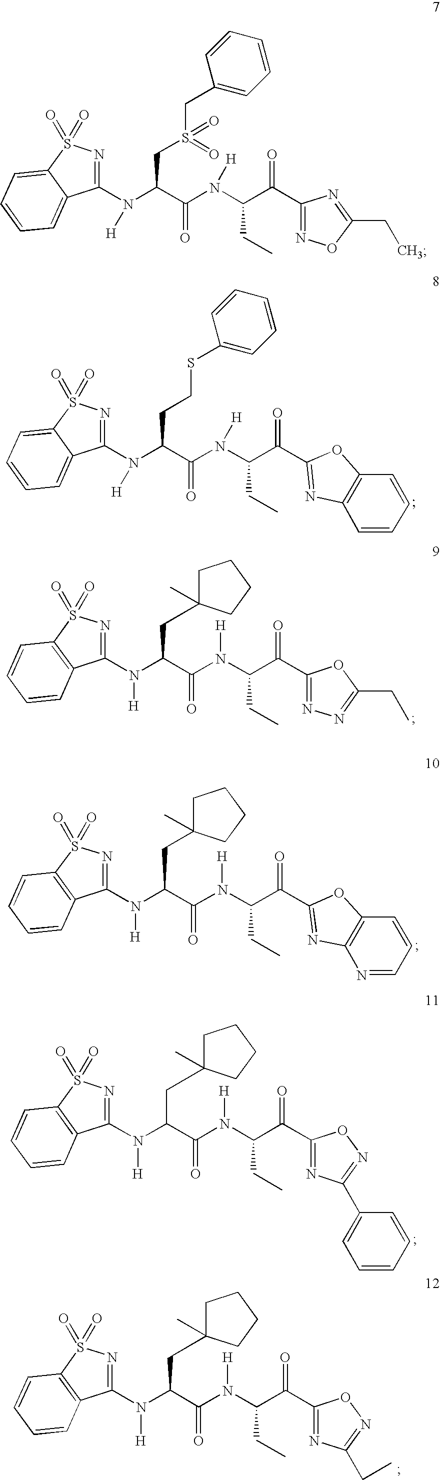 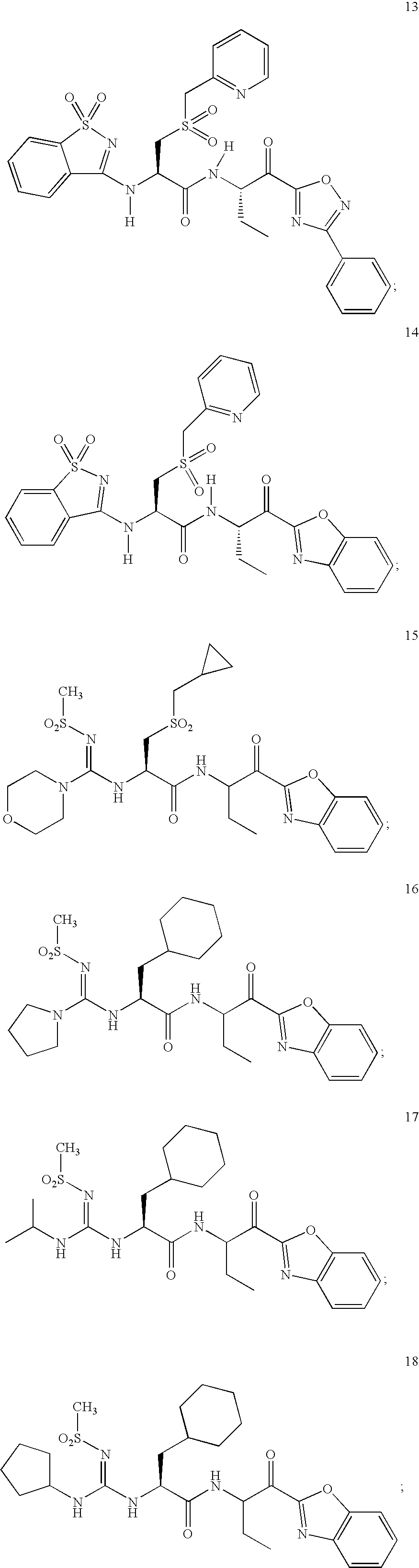 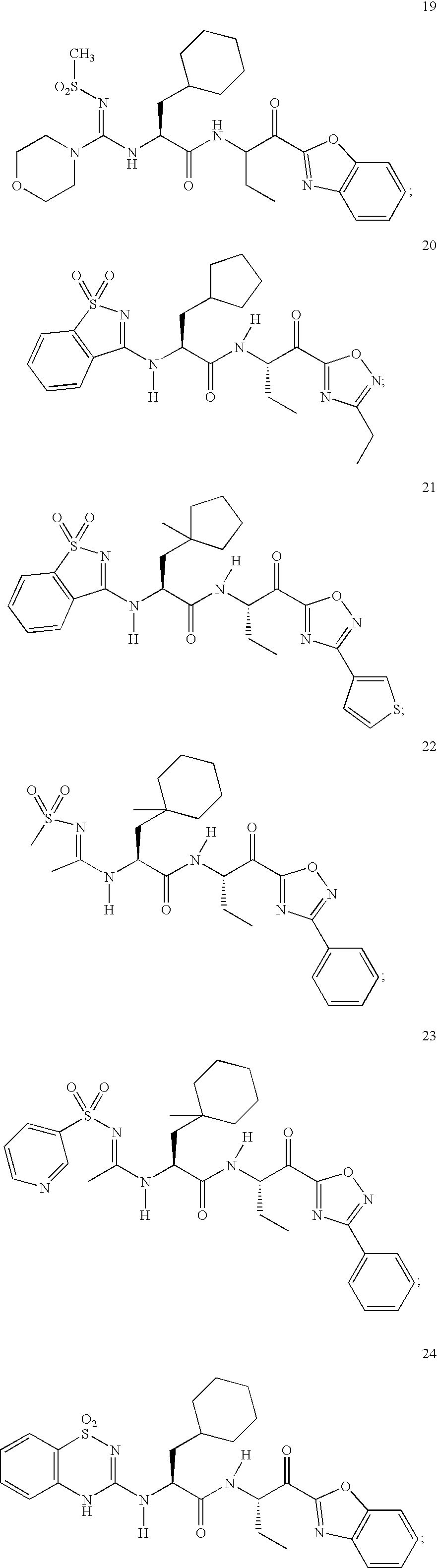 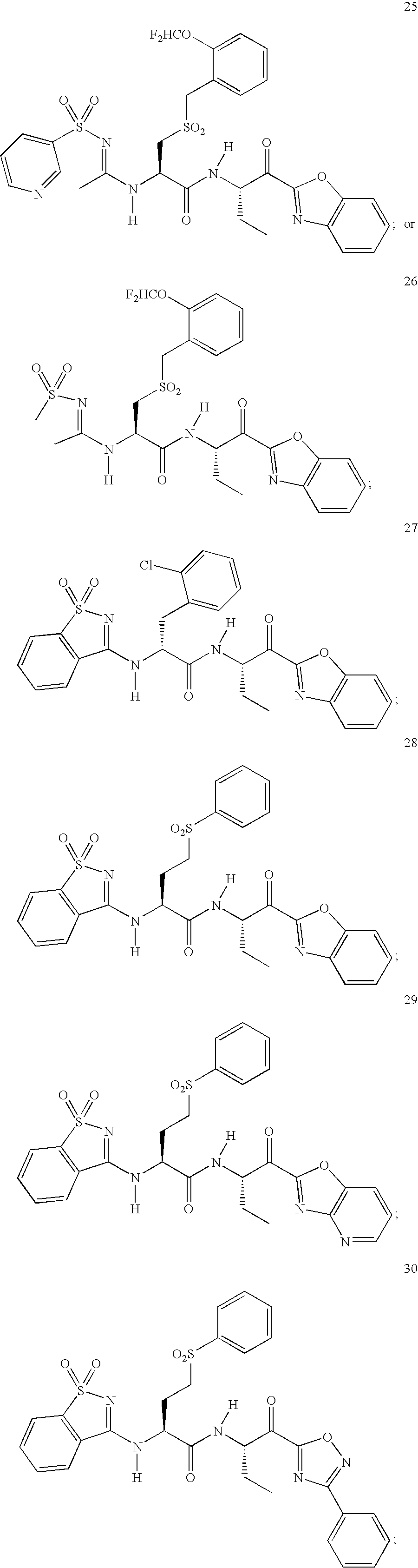 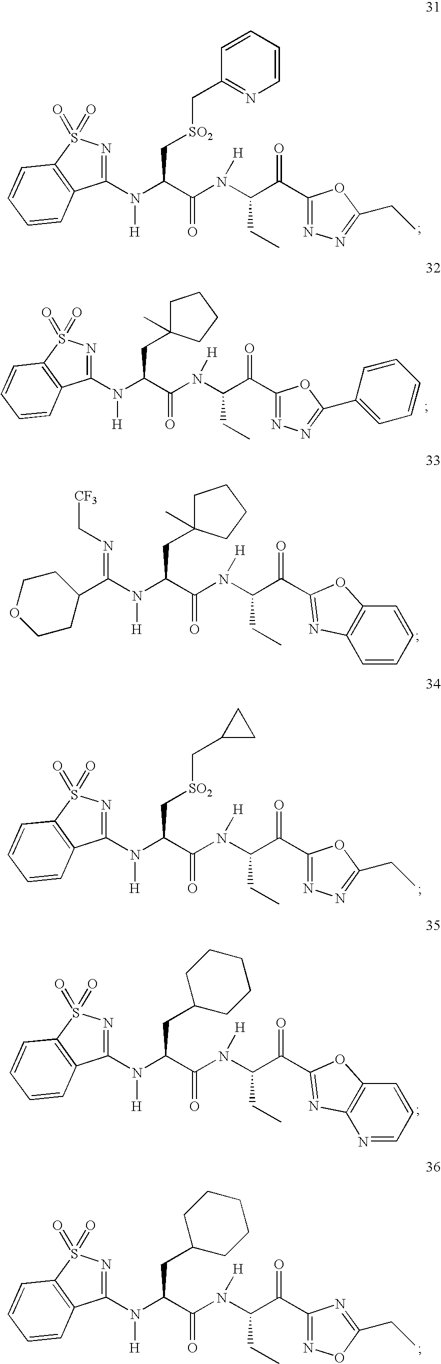 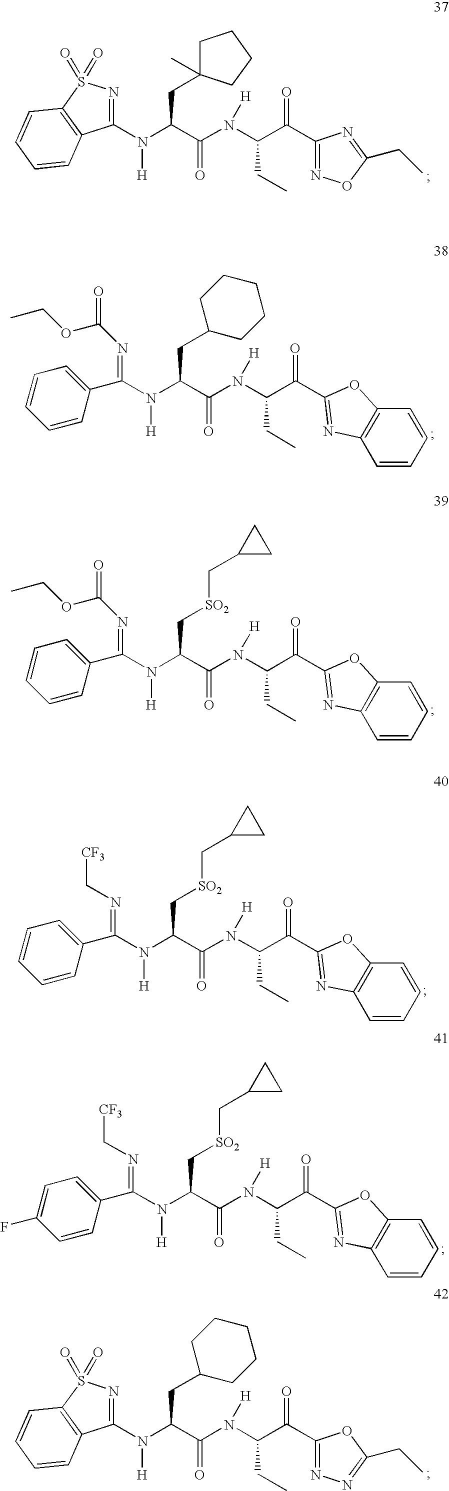 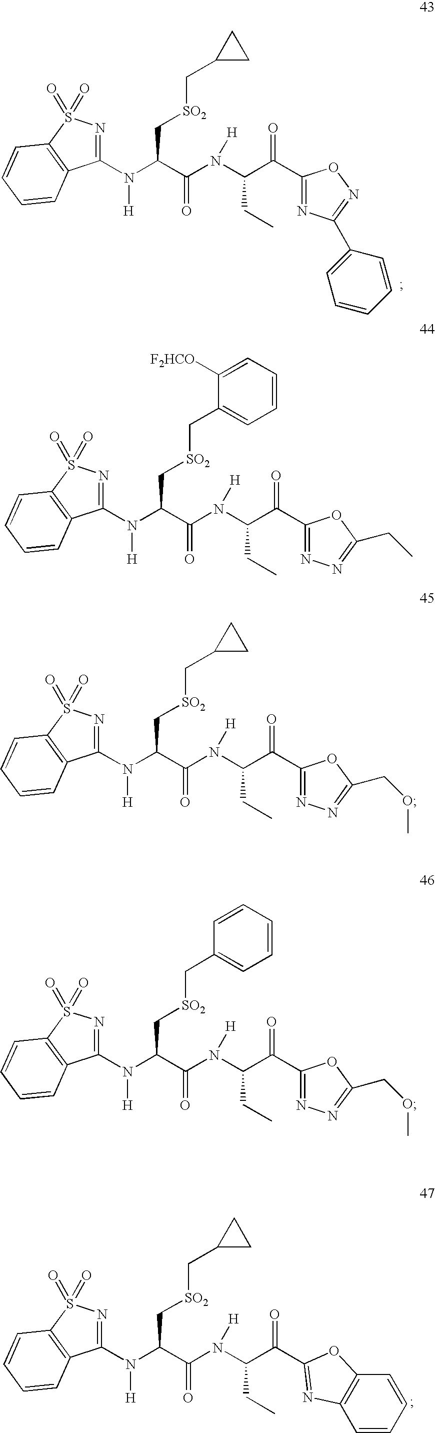 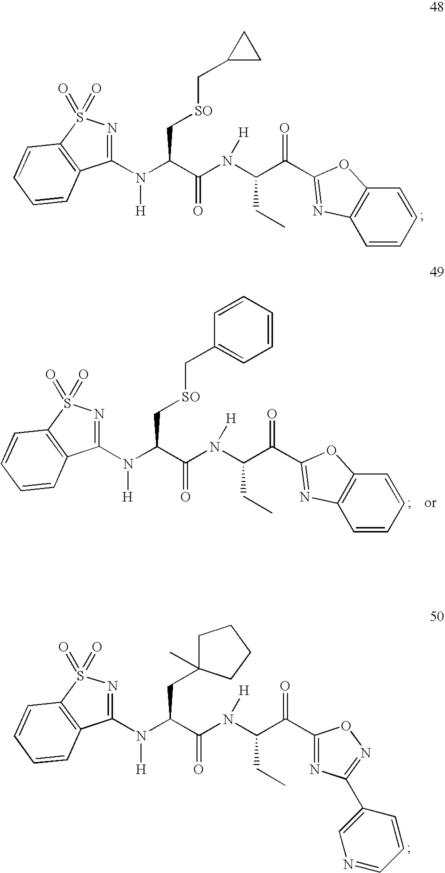
or a pharmaceutically acceptable salt thereof.

Preferably, 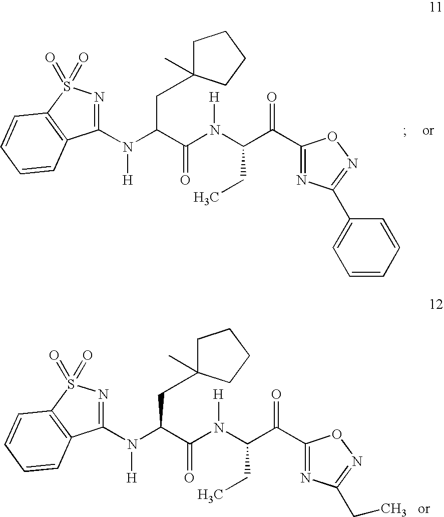
a pharmaceutically acceptable salt thereof.

In a second aspect this invention is directed to a pharmaceutical composition comprising a compound of the invention in admixture with one or more suitable excipients.

In a third aspect this invention is directed to a method for treating a disease in an animal mediated by cysteine proteases, in particular cathepsin S, which method comprises administering to the animal a therapeutically effective amount of compound of this invention.

In a fourth aspect this invention is directed to a method of treating a patient undergoing a therapy wherein the therapy causes an immune response in the patient comprising administering to the patient a compound of this invention. Preferably, the immune response is mediated by MHC class II molecules. The compound of this invention can be administered prior to, simultaneously, or after the therapy. Preferably, the therapy involves treatment with a biologic. Preferably, the therapy involves treatment with a small molecule.

Preferably, the biologic is a protein, preferably an antibody, more preferably a monoclonal antibody. More preferrably, the biologic is Remicade®, Refacto®, Referon-A®, Factor VIII, Factor VII, Betaseron®, Epogen®, Embrel®, Interferon beta, Botox®, Fabrazyme®, Elspar®, Cerezyme®, Myobloc®, Aldurazyme®, Verluma®, Interferon alpha, Humira®, Aranesp®, Zevalin® or OKT3.

Preferably, the treatment involves use of heparin, low molecular weight heparin, procainamide or hydralazine.

In a fifth aspect, this invention is directed to a method of treating immune response in an animal that is caused by administration of a biologic to the animal which method comprises administering to the animal in need of such treatment a therapeutically effective amount of a compound of this invention.

In a sixth aspect, this invention is directed to a method of conducting a clinical trial for a biologic comprising administering to an individual participating in the clinical trial a compound of this invention with the biologic.

In a seventh aspect, this invention is directed to a method of prophylactically treating a person undergoing treatment with a biologic with a compound of this invention to treat the immune response caused by the biologic in the person.

In an eigth aspect, this invention is directed to a method of determing the loss in the efficacy of a biologic in an animal due to the immune response caused by the biologic comprising administering the biologic to the animal in the presence and absence of a compound of this invention. Preferably the animal is a human.

In a ninth aspect, this invention is directed to a method of improving efficacy of a biologic in an animal comprising administering the biologic to the animal with a compound of this invention. Preferably the animal is a human.

In a tenth aspect, this invention is directed to the use of a compound of this invention for the manufacture of a medicament for combination therapy with a biologic wherein the compound of this invention treats the immune response caused by the biologic.

### DETAILED DESCRIPTION OF THE INVENTION

Definitions:

Unless otherwise stated, the following terms used in the specification and claims are defined for the purposes of this Application and have the following meanings.

“Animal” includes humans, non-human mammals (e.g., dogs, cats, rabbits, cattle, horses, sheep, goats, swine, deer, and the like) and non-mammals (e.g., birds, and the like).

“Biologic” means a therapeutic agent originally derived from living organisms for the treatment or management of a disease. Examples include, but are not limited to, proteins (recombinant and plasma derived), monoclonal or polyclonal, humanized or murine antibodies, toxins, hormones, and the like. Biologics are currently available for the treatment of a variety of diseases such as cancer, rheumatoid arthritis, and haemophilia.

“Disease” specifically includes any unhealthy condition of an animal or part thereof and includes an unhealthy condition that may be caused by, or incident to, medical or veterinary therapy applied to that animal, i.e., the “side effects” of such therapy.

“Immune response” means an immune response that prevents effective treatment of a patient or causes disease in a patient. As an example, dosing a patient with a murine antibody either as a therapy or a diagnostic agent causes the production of human antimouse antibodies that prevent or interfere with subsequent treatments. The incidence of antibody formation versus pure murine monoclonals can exceed 70%. (see Khazaeli, M. B. et al. J. Immunother. 1994, 15, pp 42-52; Dillman R. O. et al. Cancer Biother. 1994, 9, pp 17-28; and Reinsberg, J. Hybridoma. 1995, 14, pp 205-208). Additional examples of known agents that suffer from immune responses are blood-clotting factors such as factor VIII. When administered to hemophilia A patients, factor VIII restores the ability of the blood to clot. Although factor VIII is a human protein, it still elicits an immune response in hemophiliacs as endogenous factor VIII is not present in their blood and thus it appears as a foreign antigen to the immune system. Approximately 29-33% of new patients will produce antibodies that bind and neutralize the therapeutically administered factor VIII (see Lusher J. M. Semin Thromb Hemost. 2002, 28(3), pp 273-276). These neutralizing antibodies require the administration of larger amounts of factor VIII in order to maintain normal blood clotting parameters; an expensive regimen of treatment in order to induce immune tolerance (see Briet E et al. Adv. Exp. Med. Bio. 2001, 489, pp 89-97). Another immunogenic example is adenoviral vectors. Retroviral therapy remains experimental and is of limited utility. One reason is that the application of a therapeutic virus generates an immune response capable of blocking any subsequent administration of the same or similar virus (see Yiping Yang et al. J. of Virology. 1995, 69, pp 2004-2015). This ensures that retroviral therapies must be based on the transient expression of a protein or the direct incorporation of viral sequence into the host genome. Directed research has identified multiple viral neutralizing epitopes recognized by host antibodies (see Hanne, Gahery-Segard et al. J. of Virology 1998. 72, pp 2388-2397) suggesting that viral modifications will not be sufficient to overcome this obstacle. This invention will enable a process whereby an adenoviral therapy will have utility for repeated application. Another example of an immunogenic agent that elicits neutralizing antibodies is the well-known cosmetic agent Botox. Botulin toxin protein, is purified from the fermentation of Clostridium botulinum. As a therapeutic agent, it is used for muscle disorders such as cervical dystonia in addition to cosmetic application. After repeated exposure patients generate neutralizing antibodies to the toxin that results in reduced efficacy (see Birklein F. et al. Ann Neurol. 2002, 52, pp 68-73 and Rollnik, J. D. et al. Neurol. Clin. Neurophysiol. 2001, 2001(3), pp 2-4). An “immune response” also encompasses diseases caused by therapeutic agents. A specific example of this is the immune response to therapy with recombinant human erythropoietin (EPO). Erythropoietin is used to stimulate the growth or red cells and restore red blood cell counts in patients who have undergone chemotherapy or dialysis. A small percentage of patients develop antibodies to EPO and subsequently are unresponsive to both therapeutically administered EPO and their own endogenous EPO (see Casadevall, N. et al., NEJM. 2002, 346, pp 469-475). They contract a disorder, pure red cell aplasia, in which red blood cell production is severely diminished (see Gershon S. K. et. al. NEJM. 2002, 346, pp 1584-1586). This complication of EPO therapy is lethal if untreated. Another specific example is the murine antibody, OKT3 (a.k.a., Orthoclone) a monoclonal antibody directed towards CD-3 domain of activated T-cells. In clinical trials 20-40% of patients administered OKT3 produce antibodies versus the therapy. These antibodies, besides neutralizing the therapy, also stimulate a strong host immune reaction. The immune reaction is severe enough that patients with high titers of human anti-mouse antibodies are specifically restricted from taking the drug (see Orthoclone package label). A final example is a human antibody therapeutic. Humira® is a monoclonal antibody directed against TNF and is used to treat rheumatoid arthritis patients. When taken alone ˜12% of patients develop neutralizing antibodies. In addition, a small percentage of patients given the drug also contract a systemic lupus erthematosus-like condition that is an IgG-mediated immune response induced by the therapeutic agent (see Humira package label).

Another example of “immune response” is a host reaction to small molecule drugs. It is known to those skilled in the art that certain chemical structures will conjugate with host proteins to stimulate immune recognition (see Ju. C. et al. 2002. Current Drug Metabolism 3, pp 367-377 and Kimber I. et al. 2002, Toxicologic Pathology 30, pp 54-58.) A substantial portion of these host reactions are IgG mediated. Specific “immune responses” that are IgG mediated include: hemolytic anemia, Steven-Johnson syndrome and drug induced Lupus.

“Derived” means a similar agent can be traced to.

“Isomers” mean compounds of Formula (I) having identical molecular formulae but differ in the nature or sequence of bonding of their atoms or in the arrangement of their atoms in space. Isomers that differ in the arrangement of their atoms in space are termed “stereoisomers”. Stereoisomers that are not mirror images of one another are termed “diastereomers” and stereoisomers that are nonsuperimposable mirror images are termed “enantiomers” or sometimes “optical isomers”. A carbon atom bonded to four nonidentical substituents is termed a “chiral center”. A compound with one chiral center has two enantiomeric forms of opposite chirality is termed a “racemic mixture”. A compound that has more than one chiral center has 2ⁿ⁻¹ enantiomeric pairs, where n is the number of chiral centers. Compounds with more than one chiral center may exist as either an individual diastereomers or as a mixture of diastereomers, termed a “diastereomeric mixture”. When one chiral center is present a stereoisomer may be characterized by the absolute configuration of that chiral center. Absolute configuration refers to the arrangement in space of the substituents attached to the chiral center. Enantiomers are characterized by the absolute configuration of their chiral centers and described by the R— and S-sequencing rules of Cahn, Ingold and Prelog. Conventions for stereochemical nomenclature, methods for the determination of stereochemistry and the separation of stereoisomers are well known in the art (e.g., see “Advanced Organic Chemistry”, 4th edition, March, Jerry, John Wiley & Sons, New York, 1992). It is understood that the names and illustration used in this Application to describe compounds of Formula (I) encompass all possible stereoisomers.

“Pathology” of a disease means the essential nature, causes and development of the disease as well as the structural and functional changes that result from the disease processes.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Pharmaceutically acceptable salts” means salts of compounds of Formula (I)which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as acetic acid, propionic acid, hexanoic acid, heptanoic acid, cyclopentanepropionic acid, glycolic acid, pyruvic acid, lactic acid, malonic acid, succinic acid, malic acid, maleic acid, fumaric acid, tartaric acid, citric acid, benzoic acid, o-(4-hydroxybenzoyl)benzoic acid, cinnamic acid, mandelic acid, methylsulfonic acid, ethanesulfonic acid, 1,2-ethanedisulfonic acid, 2-hydroxyethanesulfonic acid, benzenesulfonic acid, p-chlorobenzenesulfonic acid, 2-naphthalenesulfonic acid, p-toluenesulfonic acid, camphorsulfonic acid, 4-methylbicyclo[2.2.2]oct-2-ene-1-carboxylic acid, glucoheptonic acid, 4,4′-methylenebis(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid and the like.

Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like.

The present invention also includes prodrugs of a compound of Formula (I). Prodrug means a compound that is convertible in vivo by metabolic means (e.g. by hydrolysis) to a compound of Formula (I).

Compounds of Formula (I) may exist as tautomers. Such tautomeric forms (individual tautomers or mixtures thereof) are within the scope of this invention. For example, a compound of Formula (I) where can tautomerize to give a compound of Formula (I) and vice versa as shown below.

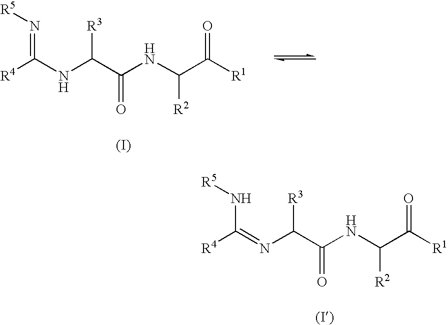

It will be recognized by a person skilled in the art that the amount of tautomers will vary based on certain conditions such as steric interactions, electronic effects of of substituents, solvent polarity, hydrogen bonding capability, temperature, pH, and the like.

“Therapeutically effective amount” means that amount which, when administered to an animal for treating a disease, is sufficient to effect such treatment for the disease.

“Treatment” or “treating” means any administration of a compound of the present invention and includes:
(1) preventing the disease from occurring in an animal which may be predisposed to the disease but does not yet experience or display the pathology or symptomatology of the disease,
(2) inhibiting the disease in an animal that is experiencing or displaying the pathology or symptomatology of the diseased (i.e., arresting further development of the pathology and/or symptomatology), or
(3) ameliorating the disease in an animal that is experiencing or displaying the pathology or symptomatology of the diseased (i.e., reversing the pathology and/or symptomatology).

“Treatment” or “treating” with respect to combination therapy i.e., use with a biologic means any administration of a compound of the present invention and includes:
(1) preventing the immune response from occurring in an animal which may be predisposed to the immune response but does not yet experience or display the pathology or symptomatology of the immune response,
(2) inhibiting the immune response in an animal that is experiencing or displaying the pathology or symptomatology of the immune response (i.e., arresting further development of the pathology and/or symptomatology), or
(3) ameliorating the immune response in an animal that is experiencing or displaying the pathology or symptomatology of the immune response (i.e., reducing in degree or severity, or extent or duration, the overt manifestations of the immune response or reversing the pathology and/or symptomatology e.g., reduced binding and presenation of antigenic peptides by MHC class II molecules, reduced activation of T-cells and B-cells, reduced humoral and cell-mediated responses and, as appropriate to the particular immune response, reduced inflammation, congestion, pain, necrosis, reduced loss in the efficacy of a biologic agent, and the like).

### General Synthetic Scheme

Compounds of this invention can be made by the methods depicted in the reaction schemes shown below.

The starting materials and reagents used in preparing these compounds are either available from commercial suppliers such as Aldrich Chemical Co., (Milwaukee, Wis.), Bachem (Torrance, Calif.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). These schemes are merely illustrative of some methods by which the compounds of this invention can be synthesized, and various modifications to these schemes can be made and will be suggested to one skilled in the art having referred to this disclosure.

The starting materials and the intermediates of the reaction may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure over a temperature range from about −78° C. to about 150° C., more preferably from about 0° C. to about 125° C. and most preferably at about room (or ambient) temperature, e.g., about 20° C.

In the reactions described hereinafter it may be necessary to protect reactive functional groups, for example hydroxy, amino, imino, thio or carboxy groups, where these are desired in the final product, to avoid their unwanted participation in the reactions. Conventional protecting groups may be used in accordance with standard practice, for examples see T. W. Greene and P. G. M. Wuts in “Protective Groups in Organic Chemistry” John Wiley and Sons, 1991. Compound of Formula (Ia) and (Ib) can be prepared by the procedures described in Schemes 1-3 below.

Compounds of Formula (I) where R¹—R⁵ are as defined in the Summary of the Invention can be prepared as shown in Scheme 1 below.

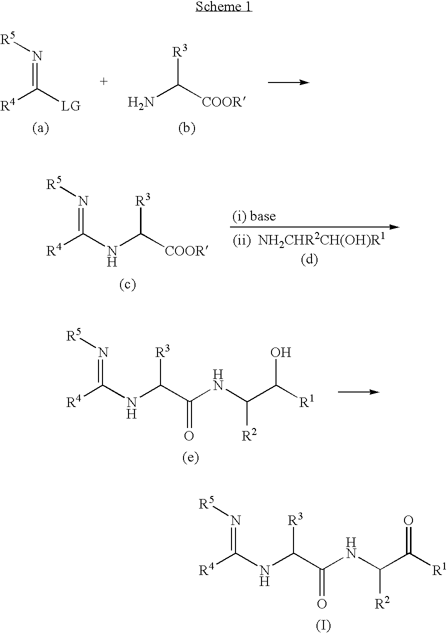

Reaction of a compound of formula (a) where LG is a leaving group such as halo with an amino acid compound of formula (b) (where R′ is hydrogen or alkyl) provides a compound of formula (c) which is then converted to a compound of Formula (I). The reaction is carried out by methods well known in the art. Some such methods are described in Dunn. A. D., Org. Prep. Proceed. Int., 1998, 30, 709; Lindstroem, S., et. al., Heterocycles, 1994, 38, 529; Katrizky, A. R., et. al., Synthesis, 1990, 561; Hontz, A. C., et. al., Org. Synth., 1963, IV, 383; and Stephen, H., J. Chem., Soc., 1957, 490.

Compounds of formula (a) are either commercially available or they can be readily prepared by methods well known in the art. Some such methods are described in working examples below. Amino acids of formula (b) are commercially available. Others can be prepared by methods well known in the art. Some such methods are described in PCT Application Publication Nos. WO 00/55144, WO 01/19816, WO 02/20485, WO 03/029200, U.S. Provisional Application No. 60/422,337, U.S. Pat. Nos. 6,353,017B1, 6,492,662B1, 353,017 B1 and 6,525,036B1, the disclosures of which are incorporated herein by reference in their entirety.

Reaction of compound (c) where R′ is hydrogen or hydrolysis of the ester group in (c) where R′ is alkyl under basic hydrolysis reaction conditions, followed by the reaction of the resulting acid with a compound of formula (d) provides a compound of formula (e). The reaction can be effected with an appropriate coupling agent (e.g., benzotriazol-1-yloxy-trispyrrolidinophosphonium hexafluorophosphate (PyBOP®), 1-(3-dimethylaminopropyl)-3-ethylcarbodiimide hydrochloride (EDCI), O-(7-azabenzotrizol-1-yl)-1,1,3,3,tetra-methyluronium-hexafluorophosphate (HATU), O-benzotriazol-1-yl-N,N,N′,N′-tetramethyl-uronium hexafluorophosphate (HBTU), 1,3-dicyclohexylcarbodiimide (DCC), or the like) and optionally an appropriate catalyst (e.g., 1-hydroxybenzotriazole (HOBt), 1-hydroxy-7-azabenzotriazole (HOAt), or the like) and non-nucleophilic base (e.g., triethylamine, N-methylmorpholine, and the like, or any suitable combination thereof) at ambient temperature and requires 5 to 10 h to complete. Suitable reaction solvents include, but are not limited to, dimethylformamide, methylene chloride, and the like. Compounds of formula (d) can be prepared by procedures described in working examples below.

Oxidation of the hydroxy group in (e) with a suitable oxidizing agent such as Oxone, Dess Martin Periodinane, TEMPO/bleach, and the like provides a compound of Formula (I).

Alternatively, a compound of Formula (I) can be prepared as shown in Scheme 2 below.

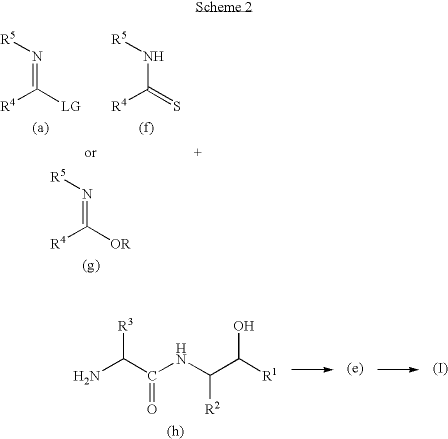

Reaction of a compound of formula (a), (f), or (g) with an amino compound of formula (h) provides a compound of formula (e) which is then converted to a compound of Formula (I) as described in Scheme 1 above. The reaction with the thione (f) is carried out in the presence of a suitable coupling agent such as 2-chloro-1-methylpyridinium iodide (see Yong, Y. F, et. al., J. Org. Chem. 1997, 62, 1540), phosgene or triphosgene (see Barton, D. H., et. al., J. Chem. Soc. Perkin Trans. I, 1982, 2085), alkyl halides (see Brand, E and Brand, F. C., Org. Synth., 1955, 3, 440), or carbodiimide (see Poss, M. A., et. al., Tet. Lett., 1992, 40, 5933).

The reaction with the imidate compound (g) is carried out under reaction conditions well known to those skilled in the art e.g., Haake, M., et. al., Synthesis, 1991, 9, 753; Dauwe, C., et al, Synthesis, 1995, 2, 171; Reid, et. al., Justus Liebigs Ann. Chem., 1966, 97, 696; and Dean N. D., and Papadopoulos, E. P. J. Het. Chem., 1982, 19, 1117.

Compounds (a), (f), and (g) are commercially available or they can be prepared by methods well known in the art e.g, see Tet. Lett., 2001, 42, 46, 8181-8184; Chem. Heterocyclo, 1972, 848-851; Chem. Heterocyclo, 1988, 337-344, PCT Application Publication No. WO 02/20485, Francesconi, I., et. al., J. Med. Chem., 1999, 42, 2260; Kurzer, F., et. al., Org. Synth. 1963, 645; Futman, A. D., U.S. Pat. No. 3,984,410, Stetter, H. and Theisen, D. H. Chem Ber., 1969, 102, 1641-42, and Ortiz, J. A., Arzneim.-Forsch./Drug Res, 1977, 47, 431-434.

Compounds of formula (h) can be prepared by reacting an N-protected amino acid of formula (b) (R′═H) with a compound of formula (d) under the coupling reaction conditions described above, followed by removal of the amino protecting group. Suitable amino protecting groups include, but are not limited to, tert-butoxycarbonyl, benzyloxycarbonyl, and the like.

Alternatively, a compound of Formula (I) where R⁴ is pyrrolidinyl, morpholinyl, isopropylamine or cyclopentylamine and is attached to the amidine carbon atom via the nitrogen atom can be prepared as shown in Scheme 3 below.

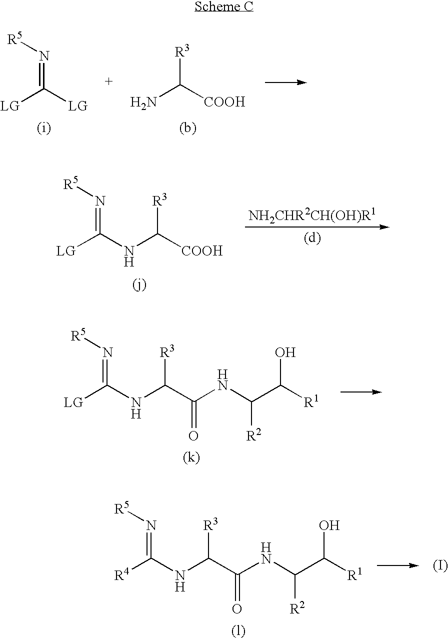

Reaction of a compound of formula (i) where LG is a leaving group, preferably methylthio, with a compound of formula (b) provides compound of formula (j). Reaction of the acid (j) with a compound of formula (d) under the reaction conditions described above provides a compound of formula (k). Reaction of (k) with isopropylamine or cyclopentylamine provides a compound of formula (l), which is then converted to a compound of Formula (I) as described above.

Other methods that can be utilized for preparing compounds of Formula (I) are described in PCT Application Publication Nos. WO 02/20485 and WO 03/029200, and U.S. Pat. No. 6,420,364, the disclosures of which are incorporated herein by reference in their entirety.

### Pharmacology and Utility

The compounds of the invention are selective inhibitors of cysteine proteases, in particular, cathepsin S, and accordingly are useful for treating diseases in which cysteine protease activity contributes to the pathology and/or symptomatology of the disease. For example, the compounds of the invention are useful in treating autoimmune disorders, including but not limited to, juvenile onset diabetes, psoriasis, multiple sclerosis, pemphigus vulgaris, Graves' disease, specifically Grave's exophthalmos, myasthenia gravis, systemic lupus erythemotasus, rheumatoid arthritis and Hashimoto's thyroiditis, allergic disorders including, but not limited to, asthma, allogeneic immune responses including, but not limited to, organ transplants or tissue grafts and endometriosis.

Cathepsin S is also implicated in disorders involving excessive elastolysis, such as chronic obstructive pulmonary disease (e.g., emphysema), bronchiolitis, excessive airway elastolysis in asthma and bronchitis, pneumonities and cardiovascular disease such as plaque rupture and atheroma. Cathepsin S is implicated in fibril formation and, therefore, inhibitors of cathepsins S are of use in treatment of systemic amyloidosis.

Preparation of Biological Agents

In practicing this invention several processes for the generation or purification of biological agents are used. Methods for preparing the biologics are well known in the art as discussed below.

Monoclonal antibodies are prepared using standard techniques, well known in the art, such as by the method of Kohler and Milstein, Nature 1975, 256:495, or a modification thereof, such as described by Buck et al. 1982, In Vitro 18:377. Typically, a mouse or rat is immunized with the MenB PS derivative conjugated to a protein carrier, boosted and the spleen (and optionally several large lymph nodes) removed and dissociated into single cells. If desired, the spleen cells may be screened (after removal of non-specifically adherent cells) by applying a cell suspension to a plate or well coated with the antigen. B-cells, expressing membrane-bound immunoglobulin specific for the antigen, will bind to the plate, and will not be rinsed away with the rest of the suspension. Resulting B-cells, or all dissociated spleen cells, are then induced to fuse with myeloma cells to form hybridomas. Representative murine myeloma lines for use in the hybridizations include those available from the American Type Culture Collection (ATCC).

Chimeric antibodies composed of human and non-human amino acid sequences may be formed from the mouse monoclonal antibody molecules to reduce their immunogenicity in humans (Winter et al. Nature 1991 349:293; Lobuglio et al. Proc. Nat. Acad. Sci. USA 1989 86:4220; Shaw et al. J. Immunol. 1987 138:4534; and Brown et al. Cancer Res. 1987 47:3577; Riechmann et al. Nature 1988 332:323; Verhoeyen et al. Science 1988 239:1534; and Jones et al. Nature 1986 321:522; EP Publication No. 519,596, published Dec. 23, 1992; and U.K. Patent Publication No. GB 2,276,169, published Sep. 21, 1994).

Antibody molecule fragments, e.g., F(ab′).sub.2, FV, and sFv molecules, that are capable of exhibiting immunological binding properties of the parent monoclonal antibody molecule can be produced using known techniques. Inbar et al. Proc. Nat. Acad. Sci. USA 1972 69:2659; Hochman et al. Biochem. 1976 15:2706; Ehrlich et al. Biochem. 1980 19:4091; Huston et al. Proc. Nat. Acad. Sci. USA 1988 85(16):5879; and U.S. Pat. Nos. 5,091,513 and 5,132,405, to Huston et al.; and U.S. Pat. No. 4,946,778, to Ladner et al.

In the alternative, a phage-display system can be used to expand the monoclonal antibody molecule populations in vitro. Saiki, et al. Nature 1986 324:163; Scharf et al. Science 1986 233:1076; U.S. Pat. Nos. 4,683,195 and 4,683,202; Yang et al. J. Mol. Biol. 1995 254:392; Barbas, III et al. Methods: Comp. Meth Enzymol. 1995 8:94; Barbas, III et al. Proc. Natl. Acad. Sci. USA 1991 88:7978.

The coding sequences for the heavy and light chain portions of the Fab molecules selected from the phage display library can be isolated or synthesized, and cloned into any suitable vector or replicon for expression. Any suitable expression system can be used, including, for example, bacterial, yeast, insect, amphibian and mammalian systems. Expression systems in bacteria include those described in Chang et al. Nature 1978 275:615, Goeddel et al. Nature 1979 281:544, Goeddel et al. Nucleic Acids Res. 1980 8:4057, European Application No. EP 36,776, U.S. Pat. No. 4,551,433, deBoer et al. Proc. Natl. Acad. Sci. USA 1983 80:21-25, and Siebenlist et al. Cell 1980 20:269.

Expression systems in yeast include those described in Hinnen et al. Proc. Natl. Acad. Sci. USA 1978 75:1929, Ito et al. J. Bacteriol. 1983 153:163, Kurtz et al. Mol. Cell. Biol. 1986 6:142, Kunze et al. J. Basic Microbiol. 1985 25:141, Gleeson et al. J. Gen. Microbiol. 1986 132:3459, Roggenkamp et al. Mol. Gen. Genet. 1986 202:302, Das et al. J. Bacteriol. 1984 158:1165, De Louvencourt et al. J. Bacteriol. 1983 154:737, Van den Berg et al. Bio/Technology 1990 8:135, Kunze et al. J. Basic Microbiol. 1985 25:141, Cregg et al. Mol. Cell. Biol. 1985 5:3376, U.S. Pat. Nos. 4,837,148 and 4,929,555, Beach et al. Nature 1981 300:706, Davidow et al. Curr. Genet. 1985 10:380, Gaillardin et al. Curr. Genet. 1985 10:49, Ballance et al. Biochem. Biophys. Res. Comnun. 1983 112:284-289, Tilburn et al. Gene 1983 26:205-221, Yelton et al. Proc. Natl. Acad. Sci. USA 1984 81:1470-1474, Kelly et al. EMBO J. 1985 4:475479; European Application No. EP 244,234, and International Publication No. WO 91/00357.

Expression of heterologous genes in insects can be accomplished as described in U.S. Pat. No. 4,745,051, European Application Nos. EP 127,839 and EP 155,476, Vlak et al. J. Gen. Virol. 1988 69:765-776, Miller et al. Ann. Rev. Microbiol. 1988 42:177, Carbonell et al. Gene 1988 73:409, Maeda et al. Nature 1985 315:592-594, Lebacq-Verheyden et al. Mol. Cell. Biol. 1988 8:3129, Smith et al. Proc. Natl. Acad. Sci. USA 1985 82:8404, Miyajima et al. Gene 1987 58:273, and Martin et al. DNA 1988 7:99. Numerous baculoviral strains and variants and corresponding permissive insect host cells from hosts are described in Luckow et al. Bio/Technology 1988 6:47-55, Miller et al. GENERIC ENGINEERING, Setlow, J. K. et al. eds., Vol. 8, Plenum Publishing, pp. 1986 277-279, and Maeda et al. Nature 1985 315:592-594.

Mammalian expression can be accomplished as described in Dijkema et al. EMBO J. 1985 4:761, Gorman et al. Proc. Natl. Acad. Sci. USA 1982 79:6777, Boshart et al. Cell 1985 41:521, and U.S. Pat. No. 4,399,216. Other features of mammalian expression can be facilitated as described in Ham et al. Meth. Enz. 1979 58:44, Barnes et al. Anal. Biochem. 1980 102:255, U.S. Pat. Nos. 4,767,704, 4,657,866, 4,927,762, 4,560,655 and Reissued U.S. Pat. No. RE 30,985, and in International Publication Nos. WO 901103430, WO 87/00195.

The production of recombinant adenoviral vectors are described in U.S. Pat. No. 6,485,958.

Botulinum toxin type A can be obtained by establishing and growing cultures of Clostridium botulinum in a fermenter and then harvesting and purifying the fermented mixture in accordance with known procedures.

Any of the above-described protein production methods can be used to provide the biologic that would benefit from the present invention.

### Testing

The cysteine protease inhibitory activity, in particular, the Cathepsin S inhibitory activities of the compounds of the invention can be determined by methods known to those of ordinary skill in the art. Suitable in vitro assays for measuring protease activity and the inhibition thereof by test compounds are known. Typically, the assay measures protease-induced hydrolysis of a peptide-based substrate. Details of assays for measuring protease inhibitory activity are set forth in Biological Examples 1-6, infra.

### Administration and Pharmaceutical Compositions

In general, a compound of the present invention will be administered in therapeutically effective amounts via any of the usual and acceptable modes known in the art, either singly or in combination with one or more therapeutic agents. A therapeutically effective amount may vary widely depending on the severity of the disease, the age and relative health of the subject, the potency of the compound used and other factors. For example, therapeutically effective amounts of a compound of compounds of the present invention may range from about 10 micrograms per kilogram body weight (μg/kg) per day to about 20 milligram per kilogram body weight (mg/kg) per day, typically from about 100 μg/kg/day to about 10 mg/kg/day. Therefore, a therapeutically effective amount for a 80 kg human patient may range from about 1 mg/day to about 1.6 g/day, typically from about 1 mg/day to about 100 mg/day. In general, one of ordinary skill in the art, acting in reliance upon personal knowledge and the disclosure of this Application, will be able to ascertain a therapeutically effective amount of a compound of the present invention for treating a given disease.

The compounds of the present invention can be administered as pharmaceutical compositions by one of the following routes: oral, systemic (e.g., transdermal, intranasal or by suppository) or parenteral (e.g., intramuscular, intravenous or subcutaneous). Compositions can take the form of tablets, pills, capsules, semisolids, powders, sustained release formulations, solutions, suspensions, elixirs, aerosols, or any other appropriate composition and are comprised of, in general, a compound of the present invention in combination with at least one pharmaceutically acceptable excipient. Acceptable excipients are non-toxic, aid administration, and do not adversely affect the therapeutic benefit of the active ingredient. Such excipient may be any solid, liquid, semisolid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk, and the like. Liquid and semisolid excipients may be selected from water, ethanol, glycerol, propylene glycol and various oils, including those of petroleum, animal, vegetable or synthetic origin (e.g., peanut oil, soybean oil, mineral oil, sesame oil, and the like). Preferred liquid carriers, particularly for injectable solutions, include water, saline, aqueous dextrose and glycols.

The amount of a compound of the present invention in the composition may vary widely depending upon the type of formulation, size of a unit dosage, kind of excipients and other factors known to those of skill in the art of pharmaceutical sciences. In general, a composition of a compound of the present invention for treating a given disease will comprise from 0.01% w to 10% w, preferably 0.3% w to 1% w, of active ingredient with the remainder being the excipient or excipients. Preferably the pharmaceutical composition is administered in a single unit dosage form for continuous treatment or in a single unit dosage form ad libitum when relief of symptoms is specifically required. Representative pharmaceutical formulations containing a compound of the present invention are described in working examples below.

### EXAMPLES

The present invention is further exemplified, but not limited by, the following examples that illustrate the preparation of compounds of Formula (I) according to the invention.

### Reference A

### Synthesis of 2(S)-(tert-butoxycarbonyl)amino-1-(oxazolo[4.5-b]pyridin-2-yl)butan-1-ol

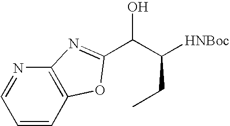 Step 1

A mixture of 2-amino-3-hydroxypyridine (11 g, 100 mmol), triethylorthoformate (80 mL) and p-toluenesulfonic acid (61 mg) was heated at 140° C. for 8 h. Excess triethylorthoformate was removed under vacuum and oxazolo[4.5-b]pyridine was crystalized from ethyl acetate (9 g).

Step 2

In a clean roundbottom flask equipped with stir bar was placed oxazolo[4,5-b]pyridine (600 mg, 5 mmol) in THF (30 mL) and the reaction mixture was cooled to 0° C. under N₂ atomosphere. Isopropylmagnesium chloride (2M in THF, 2.5 mL, 5 mmol) was added. After stirring for 1 h at 0° C., 2(S)-(tert-butoxycarbonyl)aminobutyraldehyde (573 mg, 3 mmol) in THF (20 mL) was added. The ice bath was removed and the reaction mixture was allowed to warm to room temperature. After 2 h, the reaction mixture was quenched with saturated ammonium chloride solution and concentrated to dryness. The residue was extracted with EtOAc, washed with brine, dried with anhyd. MgSO₄, filtered and concentrated. The crude product was purified by chromatograph to yield 383 mg of the title compound.

H¹ NMR (DMSO-d₆): δ8.42 (m, 1H), 8.18 (m, 1H), 7.3(m, 1H), 6.8-6.6 (dd, d, 1H, OH, diastereomer), 6.3-6.02 (d, d, 1H, NH, diastereomer), 4.82-4.5 (m,m, 1H, diastereomer), 1.8-1.3 (m, 2H), 1.2-1.05 (s,s, 9H, diastereomer), 0.89 (m, 3H). MS: 306.2 (M−1), 308.6 (M+1).

### Reference B

### Synthesis of 2(S)-amino-1-(3-phenyl-[1.2.4]oxadiazol-5-yl)-butan-1-ol

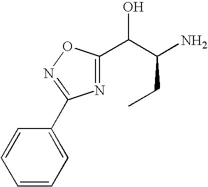

3-tert-Butoxycarbonylamino-2-hydroxypentanoic acid (500 mg, 2.14 mmol) was combined with EDC (600 mg, 3.14 mmol), HOBt (600 mg, 3.92 mmol), and N-hydroxy-benzamidine (292 mg, 2.14 mmol). Dichloromethane (10 mL) was added and then 4-methylmorpholine (1 mL). The reaction mixture was stirred at ambient temperature for 16 h. After dilution with ethyl acetate (200 mL), the solution was washed with water (30 mL), saturated aqueous NaHCO₃ solution and brine, dried with MgSO₄ and evaporated under vacuum. The crude product was dissolved in pyridine (10 mL) and heated at 80° C. for 15 h. The pyridine was evaporated under vacuum and the residue was purified by flash chromatography on silica gel (eluent: ethyl acetate) to yield 2(S)-tert-butoxycarbonylamino-1-(3-phenyl-[1.2.4]oxadiazol-5-yl)butan-1-ol (290 mg, 0.83 mmol). 2(S)-tert-butoxycarbonylamino-1-(3-phenyl-[1.2.4]oxadiazol-5-yl)-butan-1-ol (145 mg, 0.41 mmol) was dissolved in CH₂Cl₂ (4 mL) and TFA (4 mL) was added. After stirring for 1 h, the reaction mixture was evaporated to dryness to yield the title compound.

Following the procedure described above but substituting N-hydroxypropamidine for N-hydroxybenzamidine provided 2(S)-amino-1-(3-ethyl-[1.2.4]oxadiazol-5-yl)-butan-1-ol.

### Reference C

### Synthesis of 2(S)-amino-1-(2-methoxymethyl-[1.3.4]oxadiazol-5-yl)butan-1-ol

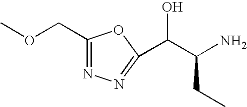 Step 1

(S)-(+)-2-Amino-1-butanol (50 g, 561 mmol) in a mixture of water and dioxane (200 mL of water and 200 mL dioxane) was cooled to 0° C. and NaOH (26.9 g, 673 mmol) and di-tert-butyldicarbonate (146.96 g, 673 mmol) were added. After the addition, the reaction mixture was allowed to warm to room temperature and stirred for 2 h. After removing the dioxane, the residue was extracted with EtOAc, then washed with brine and dried with anhydrous MgSO₄, filtered and concentrated. Without further purification, the crude 2(S)-Boc-amino-1-butanol (120 g) was used in the next step.

Step 2

A solution of oxalyl chloride (40.39 g, 265 mmol) in CH₂Cl₂ (700 mL) was stirred and cooled to −60° C. Dimethylsulfoxide (51.7 g, 663 mmol) in CH₂Cl₂ (100 mL) was added dropwise. After 10 min, a solution of 2(S)-Boc-amino-1-butanol (50 g, 265 mmol) in CH₂Cl₂ (100 mL) was added dropwise at −70° C. The reaction mixture was allowed to warm to −40° C. for 10 min and then cooled to −70° C. again. A solution of triethylamine (74.9 g, 742 mmol) in CH₂Cl₂ (100 mL) was added and the reaction mixture was allowed to warm to room temperature over 2 h. Saturated sodium dihydrogen phosphate (100 mL) was added and then the organic layer was washed with brine and dried over MgSO₄. The solvent was removed to yield 45 g of 2(S)-Boc-aminobutyraldehyde(1-formylpropyl)carbamic acid tert-butyl ester.

Step 3

A mixture of methyl methoxyacetate (52 g, 500 mmol), hydrazine hydrate (30 mL) was heated to reflux for 8 h. Excess hydrazine and water were removed under vacuum. The residue was extracted with n-butanol, dried with Na₂SO₄. Excess n-butanol was removed to yield 45 g of hydrazide.

Step 4

A mixture of above hydrazide (45 g), triethylorthoformate (146 mL) and p-toluenesulfonic acid (61 mg) was heated at 140° C. for 8 h. Excess triethylorthoformate was removed under vacuum. The product was purified by silica gel column chromatography to yield 4.6 g of 2-methoxymethyl-[1.3.4]-oxadiazole.

Step 5

To a stirred solution of 2-methoxymethyl-[1.3.4]-oxadiazole (4.6 g, 40 mmol) in THF (100 mL) was added n-BuLi (1.6 M solution in 25.2 mL of hexane) dropwise under N₂ at −78° C. After 1 h, MgBr.Et₂O (10.4 g, 40.3 mmol) was added and the reaction mixture was allowed to warm to −45° C. for 1 h before being treated with 2(S)-Boc-aminobutyraldehyde(1-formylpropyl)carbamic acid tert-butyl ester (5.28 g, 28.25 mmol) in THF (20 mL). The reaction mixture was stirred for 1 h, quenched with saturated NH₄Cl, and extracted with ethyl acetate. The organic layer was washed with brine, dried with MgSO₄ and concentrated. The residue was purified by silica gel column chromatography to yield 2(5)-Boc-amino-1-(5-methoxymethyl-[1.3.4]-oxadiazol-2-yl)-1-propanol butanol (500 mg).

Step 6

2(S)-Boc-amino-1-(5-methoxymethyl-[1.3.4]-oxadiazol-2-yl)-1-propanol butanol (500 mg, 1.66 mmol), and CH₂Cl₂ (5 mL) were mixed and TFA (0.5 mL) was added at room temperature. After stirring for 1 h, the solvent and excess TFA were removed under vacuum to the title compound as the TFA salt (340 mg).

### Reference D

### Synthesis of 2(S)-amino-1-(2-phenyl-[1.3.4]oxadiazol-5-yl)butan-1-ol

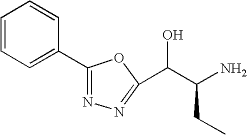 Step 1

A mixture of the benzoic hydrazide (22.5 g, 165 mmol), triethylorthoformate (150 mL) and p-toluenesulfonic acid (300 mg) was heated at 120° C. for 12 h. Excess triethylorthoformate was removed under vacuum and the residue was purified by silica gel column chromatography to produce 2-phenyl-[1.3.4]-oxadiazole (14.5 g).

Step 2

To a stirred solution of the 2-phenyl-[1.3.4]oxadiazole (10 g, 68.5 mmol) in THF (100 mL) was added n-BuLi (1.6 M solution in 42.8 mL of hexane) dropwise under N₂ at −78° C. After 1 h, MgBr.Et₂O (17.69 g, 68.5 mmol) was added and the reaction mixture was allowed to warm to −45° C. for 1 h before being treated with 2(S)-Boc-aminobutyraldehyde (7.8 g, 41 mmol) in THF (20 mL). The reaction mixture was stirred for 1 h, quenched with saturated NH₄Cl, and extracted with ethyl acetate. The organic layer was washed with brine, dried with MgSO₄ and concentrated. The residue was purified by silica gel column chromatography to yield 2-[2(S)-Boc-amino-1-hydroxybutyl]-5-phenyl-[1.3.4]-oxadiazole (9.7 g).

Step 3

2-[2(8)-Boc-amino-1-hydroxybutyl]-5-phenyl-[1,3,4]-oxadiazole (505 mg, 1.5 mmol) and CH₂Cl₂ (5 mL) were mixed and TFA (1 mL) was added at room temperature. After stirring for 1 h, the solvent and excess TFA were removed under vacuum to produce 530 mg of the title compound as the TFA salt.

### Reference E

### Synthesis of 2(S)-amino-1-oxazolo[4.5-b]pyridin-2-ylbutan-1-ol

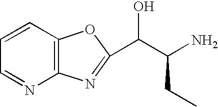 Step 1

A mixture of 2-amino-3-hydroxypyridine (25 g, 227 mmol), triethylorthoformate (75 mL) and p-toluenesulfonic acid (61 mg) was heated at 140° C. for 8 h. Excess triethylorthoformate was removed under vacuum. The product was crystallized from ethyl acetate to yield 22.5 g of oxazolo[4.5-b]pyridine.

Step 2

To a stirred solution of the oxazolo[4.5-b]pyridine (12 g, 100 mmol) in THF (300 mL) was added n-BuLi (1.6 M solution in 62.5 mL of hexane) drop wise under N₂ at −78° C. After 1 h, MgBr.Et₂O (25.8 g, 100 mmol) was added and the reaction mixture was allowed to warm to −45° C. for 1 h before being treated with 2(S)-Boc-aminobutylaldehyde (11.46 g, 60 mmol) in THF (50 mL). The reaction mixture was stirred for 1 h, quenched with saturated NH₄Cl, and extracted with ethyl acetate. The organic layer was washed with brine, dried with MgSO₄ and concentrated. The residue was purified by silica gel column chromatography to yield 2(S-Boc-amino-1-(oxazolo[4.5-b]pyridin-2-yl)-1-butanol (14.1 g).

Step 3

2(S)-Boc-amino-1-(oxazolo[4.5-b]pyridin-2-yl)-1-butanol (311 mg, 1 mmol) and CH₂Cl₂ (5 mL) were mixed and TFA (1 mL) was added at room temperature. After stirring for 1 h, the solvent and excess TFA were removed under vacuum to produce 355 mg of the title compound as the TFA salt.

### Reference F

### Synthesis of 2(S)-amino-1-benzoxazol-2-ylbutan-1-ol hydrochloride

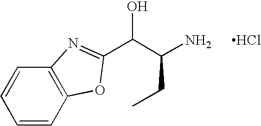 Step 1

To a solution of benzoxazole (28.6 g, 240 mmol) in toluene (150 mL) was added to a 2 M solution of isopropylmagnesium chloride in THF (120 mL, 240 mmol) at about −4° C. The red-brown mixture was stored at ca −4° C. and used as needed.

Step 2

To a solution of 2(S)-Boc-aminobutanol (50 g; 264 mmol) in dichloromethane (500 mL) and water (350 mL) were added at 20° C. TEMPO (0.01 eq), sodium bromide (1 eq) and sodium hydrogencarbonate (3 eq). The reaction mixture was stirred at 0° C. and diluted bleach (1.3 eq, 450 mL) was added over 40 min. The reaction mixture was stirred for 30 min. at 0° C. and then quenched with aq. thiosulfate. After decantation and extractions (dichloromethane), the organic phase was washed with brine, dried and concentrated in vacuo to dryness, giving 2(S)-tert-butoxycarbonylaminobutyraldehyde as a low-melting solid (38.1 g; yield: 77%).

Step 3

A solution of 2(S)-tert-butoxycarbonyl)aminobutyraldehyde (30 g, 160 mmol) in toluene (150 mL) was added over 30 min at −5° C. to a solution of Grignard reagent of benzoxazole (prepared as described in Step 1 above). The reaction mixture was stirred for 0.5 h at 0° C., then 2.5 h at RT. Quenching with 5% aq. acetic acid, washings with 5% aq. sodium carbonate, then brine and concentration to dryness gave crude 2(S)-tert-butoxycarbonyl-amino-1-benzoxazol-2-ylbutan-1-ol. The residue was diluted with toluene, and silica gel was added. The slurry was filtered. Elution by toluene removed the non-polar impurities. Then an 8/2 mixture of toluene and ethyl acetate was used to desorb the 2(S)-tert-butoxycarbonyl amino-1-benzoxazol-2-ylbutan-1-ol.

Step 4

To a solution of 2(S)-tert-butoxycarbonyl amino-1-benzoxazol-2-ylbutan-1-ol (26.3 g, 86 mmol) in isopropanol (118 mL) at 20-25° C. was added trimethylchlorosilane (1.4 eq). The solution was stirred for 5 h at 50° C. Concentration of the reaction mixture to 52 mL followed by addition of isopropyl ether (210 mL), filtration and drying under vacuum afforded the title compound as a grey solid (16.4 g, yield=79%; mixture of diastereomers).

### Reference G

### Synthesis of 2(S)-Boc-amino-1-(2-ethyl-[1.3.4]oxadiazol-2-yl)butan-1-ol

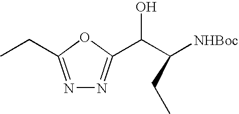 Step 1

A mixture of the formic hydrazide (60 g, 1 mole), triethylorthopropionate (176.26 g, 1 mole) and p-toluenesulfonic acid (250 mg) was heated at 120° C. for 12 hours. The ethanol was removed under vacuum and the residue was distilled under vacuum to yield 24 g of ethyl-[1.3.4]-oxadiazole.

Step 2

To a stirred solution of the ethyl-[1.3.4]-oxadiazole (4.66 g, 48 mmol) in THF (50 mL) was added n-BuLi (1.6M solution in 30 mL of hexane) dropwise under N₂ at −78° C. After 1 hour, MgBr.Et₂O (12.38 g, 48 mmol) was added and the reaction mixture was allowed to warm to −45° C. for 1 hour before being treated with 2(S)-tert-butoxycarbonyl)aminobutyraldehyde (3.2 g, 24 mmol) in THF (20 mL). The reaction mixture was stirred for 1 hour, quenched with saturated NH₄Cl, and extracted with ethyl acetate. The organic layer was washed with brine, dried with MgSO₄ and concentrated. The residue was purified by silica gel column chromatography to yield the title compound (2.13 g).

¹NMR (DMSO-δ): 6.65-6.52 (1H, d, d, J=9.2 Hz, J=9.2 Hz, NH, diastereomer), 6.14, 5.95 (1H, d, d, J=5.6 Hz, J=5.6 Hz, OH, diastereomer), 4.758-4.467 (1H, m, diastereomer), 3.7-3.55 (1H, m), 2.8 (2H, q), 1.33(12H, t), 1.25-1.21 (2H, m), 0.82 (3H, m). MS: 284.1 (M−1), 286

### Reference H

### Synthesis of thiophene-2-carbothioic acid (2,2,2-trifluoroethyl)amide

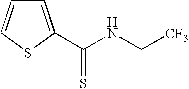 Step 1

Thiophene-2-carboxylic acid was coupled to trifluoroethyl amine by the procedure described in Example 1, Step 2 below except substituting HOBt with HBTU to give thiophene-2-carboxylic acid (2,2,2-trifluoroethyl)amide.

Step 2

To thiophene-2-carboxylic acid (2,2,2-trifluoroethyl)amide (2.8 g, 13.28 mmol, 1.0 equiv.) in toluene (100 mL) was added Lawesson's reagent (2.71 g, 6.69 mmol, 0.5 equiv.) The solution was stirred at 100° C. for 3 h. The solvent was removed in vacuo and the resulting residue was purified by flash chromatography (5% EtOAc/hexanes as eluent) to afford the title compound (1.4 g) as a yellow solid. MS=225.9 (M+1).

Proceeding as described above, but substituting thiophene-2-carboxylic acid with commercially available starting materials, the following compounds were prepared:

Phenyl-2-carbothioic acid (2,2,2-trifluoroethyl)amide; MS=220 (M+1)

4-Fluorophenylcarbothioic acid (2,2,2-trifluoroethyl)amide; MS=238 (M+1); and

Tetrahydropyran-4-carbothioic acid (2,2,2-trifluoroethyl)amide; MS=225.8 (M−1).

### Reference I

### Synthesis of 1,1-dioxo-1,2-dihydro-1λ⁶-thieno[2,3-d]isothiazol-3-one

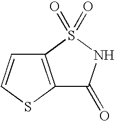 Step 1

Methyl 4-(chlorosulfonyl)thiophene-3-carboxylate (5 g, 20.75 mmol) was dissolved in methylene chloride (50 mL), the solution was cooled to 0° C. and ammonia gas (1.1 g, 64.7 mmol) was introduced during 20 min. After a further 2 h of stirring, the reaction mixture was washed to neutrality with 10% aqueous hydrochloric acid and then with brine. After concentration of the solvent, crude methyl 4-sulfamoylthiophene-3-carboxylate was obtained which was recrystallized from ethanol to yield 2.7 g of methyl 4-sulfamoylthiophene-3-carboxylate. MS: 220 (M−1), 221.9 (M+1), 243.8 (M+Na).

Step 2

A mixture of methyl 4-sulfamoylthiophene-3-carboxylate (2.7 g, 12.2 mmol), methanol (12 mL), and a 25% methanolic solution of sodium methylate (3.6 mL) was refluxed for 48 h. The reaction mixture was cooled to room temperature and acidified with concentrated hydrochloride acid, and the precipitated product was collected and washed with water. Recrystallized of the crude product from water, yielded 400 mg of the title compound. MS: 187.8 (M−1), 189.5 (M+1). ¹H NMR (DMSO-d₆): 8.34 (d, J=4.4 Hz, 1H), 7.705 (d, J=4.8 Hz, 1H).

### Reference J

### Synthesis of 1-cyclopentyl-3-methylsulfonylthiourea

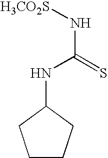

To methylsulfonyl isothiocyanate (175 mg, 0.128 mmol, 1.0 equiv.) (prepared according to a literature procedure described in J. Org. Chem. 1967, p 340) in benzene (1 mL) was added cyclopentylamine (130 mg, 1.53 mmol 1.2 equiv.) and the mixture was heated to 80° C. in a microwave instrument. Upon cooling, 1-cyclopentyl-3-methylsulfonylthiourea precipitated as brown needles (210 mg).

### Reference K

### Synthesis of 2(R)-tert-butoxycarbonylamino-3-cyclopropylmethanesulfonylpropionic acid

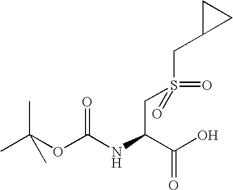 Step 1

Sodium hydroxide (2.16 g, 54 mmol) was dissolved in water (27 mL) and the solution added to a suspension of 2(R)-tert-butoxycarbonylamino-3-mercaptopropionic acid (8.2 g, 37 mmol) in methanol (54 mL). After a clear solution had formed bromomethylcyclopropane (5 g, 37 mmol) was added and the resulting reaction mixture stirred for three days. Methanol was removed under reduced pressure. The residue was treated with 1M hydrochloric acid (200 mL) and then extracted with dichloromethane. The combined organic phases were washed with brine and dried with magnesium sulfate. The solvent was evaporated under reduced pressure to give 2-tert-butoxycarbonylamino-3-cyclopropylmethylsulfanylpropionic acid (7.94 g).

Step 2

Sodium hydroxide (2.32 g, 58 mmol) was dissolved in water (27 mL) and 2-tert-butoxycarbonylamino-3-cyclopropylmethylsulfanyl-propionic acid (7.94 g, 29 mmol) was added. A solution of Oxone™ in water (100 mL) was added slowly. The pH was adjusted to 3 by addition of sodium bicarbonate and the reaction mixture stirred for 30 minutes and extracted with ethyl acetate. The combined organic phases were washed with brine and dried with magnesium sulfate. The solvent was removed to yield 2(R)-tert-butoxycarbonylamino-3-cyclopropylmethanesulfonylpropionic acid (4.64 g, 15 mmol, 31%).

### Reference L

### Synthesis of 2(S)-amino-N-[1(S)-(benzoxazol-2-ylhydroxymethyl)propyl]-3-(1-methyl-cyclopentyl)propionamide

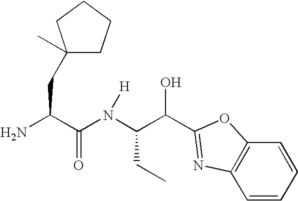 Step 1

1-Methylcyclopentanol (20 g, 0.2 mol) was added to hydrobromic acid (40 mL) at room temperature. After stirring for 1 h, the solution was extracted with hexane and the hexane was washed with brine and dried with magnesium sulfate. After concentration of the organic layer, 20.5 g of 1-methylcyclopentyl bromide was obtained.

Step 2

Tributyltin hydride (37.8 g, 130 mmol) was added at reflux to a 500 ml of flask charged with benzene (200 mL) was added Z-dehydro-Ala-OH (15 g, 64 mmol), 1-methylcyclopentanyl-bromide (20.5 g) and AIBN (1.9 g). After 2 h, the solvent was removed and the residue was purified by column chromatograph to yield 7.9 g of 2-benzyloxycarbonylamino-3-(1-methyl-cyclopentyl)propionic acid methyl ester.

Step 3

2-Benzyloxycarbonylamino-3-(1-methylcyclopentyl)propionic acid methyl ester (7.6 g, 23.8 mmol) was dissolved in a mixture of acetonitrile (82 mL) and 0.2 M aqueous NaHCO₃ (158 mL) and Alcalase 2.4 L (1.1 mL) was added and the reaction mixture was stirred vigorously for 8 h. The reaction mixture was then evaporated at 30° C. to remove acetonitrile, and the aqueous residue was washed with ether. The ethereal layer was concentrated to yield 1.9 g of 2(R)-benzyloxycarbonylamino-3-(1-methylcyclopentyl)propionic acid methyl ester. The aqueous phase was filtered with Celite, the pH was adjusted to 3 with 6 N HCl, and the solution was extracted with ethylacetate. The ethyl acetate layer was dried and evaporated to yield 1.4 g of 2(S)-benzyloxycarbonylamino-3-(1-methylcyclopentyl)propionic acid.

Step 4

To a stirred mixture of 2(8)-benzyloxycarbonylamino-3-(1-methylcyclopentyl)propionic acid (560 mg, 1.84 mmol), 2(S)-amino-1-benzoxazol-2-ylbutan-1-ol (378 mg, 1.84 mmol), and HOBt (338 mg, 2.2 mmol) in CH₂Cl₂ (10 mL) were added EDC (533 mg, 2.76 mmol) and N-methylmorpholine (373 mg) at room temperature. After stirring for 14 h, the reaction mixture was extracted with ethyl acetate. The organic layer was washed with saturated NaHCO₃, brine, dried with MgSO₄ and concentrated. Purification with column chromatograph yielded 600 mg of [1-[1(S)-(benzoxazol-2-ylhydroxymethyl)propylcarbamoyl]-2(S)-(1-methylcyclopentyl)-ethyl]carbamic acid benzyl ester.

Step 5

Pd/C (5%) (60 mg) was added to a solution of [1-[1(S)-(benzoxazol-2-ylhydroxymethyl)-propylcarbamoyl]-2(S)-(1-methylcyclopentyl)ethyl]carbamic acid benzyl ester (600 mg) in EtOH (30 mL) and the reaction mixture was stirred under hydrogen atmosphere (50 psi) for 2 h. The catalyst was removed by filtration and the filtrate was concentrated to yield 430 mg of the title compound. MS: 358.2 (M−1). 360.1 (M+1), 382.0(M+Na).

### Example 1

### Synthesis of 2(R)-(1,1-dioxo-2,3-dihydro-1H-λ⁶-benzo[d]isothiazol-3-yl)-N-[1(S)-(3-ethyl-[1.2.4]oxadiazol-5-ylcarbonyl)propyl]-3-(1-methylcyclopentyl)propionamide

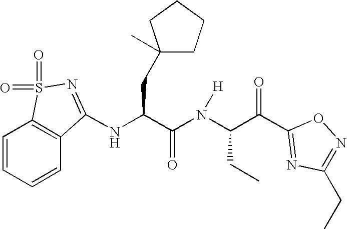 Step 1

2(S)-Amino-3-cyclopentyl-3-methylpropionic acid hydrobromide (1 g, 3.97 mmol) was dissolved in 1N aq. sodium hyroxide (10 mL) and dioxane (5 mL) and cooled in an ice bath. 3-Chlorobenzo[d]isothiazole-1,1-dioxide (0.804 g, 4 mmol), prepared by the procedure described in David, F. A., J. Org. Chem., 1990, 55, 1254, was added. The reaction mixture was allowed to warm to room temperature and then acidified with 1N HCl and the product was isolated with ethyl acetate. Crystallization from ethyl acetate-hexane mixture gave 3(S)-cyclopentyl-2-(1,1-dioxobenzo[d]isothiazol-3-ylamino)-3-methylpropionic acid (1.5 g).

Step 2

A mixture of 3-cyclopentyl-2(S)-(1,1-dioxobenzo[d]isothiazol-3-ylamino)-3-methylpropionic acid (0.168 g, 0.5 mmol), 2(S)-amino-(3-ethyl-[1.2.4]-oxadiazol-5-yl)butan-1-ol (0.093 g, 0.5 mmol), HOBt (0.092 g, 0.6 mmol), EDC (0.145 g, 0.75 mmol), NMM (0.202 g, 2 mmol), and methylene chloride (5 mL) was stirred at room temperature. The product was isolated by dilution of the reaction mixture with water and extraction with ethyl acetate to give 2(R)-(1,1-dioxo-2,3-dihydro-1H-λ⁶-benzo[d]isothiazol-3-yl)-N-{1(S)-[(3-ethyl-[1.2.4]oxadiazol-5-yl)-hydroxymethyl]propyl}-3-(1-methylcyclopentyl)propionamide.

Step 3

A solution of 2(R)-(1,1-dioxo-2,3-dihydro-1H-λ⁶-benzo[d]isothiazol-3-yl)-N-{1(S)-[(3-ethyl-[1.2.4]oxadiazol-5-yl)-hydroxymethyl]propyl}-3-(1-methylcyclopentyl)propionamide (0.25 g, 0.5 mmol) in methylene chloride (5 mL) was treated with Dess-Martin periodinane (0.254 g, 0.6 mmol) at room temperature. The reaction was followed by HPLC. The reaction mixture was quenched with aq. sodium thiosulfate. Water-ethyl acetate work up, followed by column chromatography gave the title compound (0.049 g). M. pt. 202-204° C. MS 500.4 (M−1) and 502.2 (M+1).

### Example 2

### Synthesis of ({1-[1(S)-(benzooxazol-2-ylcarbonyl)propylcarbamoyl]-2(S)-cyclohexylethylamino}phenylmethylene)carbamic acid ethyl ester

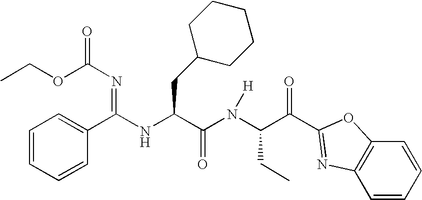 Step 1

A mixture of N-Boc-cyclohexylalanine (1.8 g, 6.58 mmol), 2(S)-amino-1-benzoxazol-2-ylbutan-1-ol (1.6 g, 6.58 mmol), EDC (1.65 g, 8.5 mmol), HOBt (1.21 g, 7.9 mmol), NMM (1.42 mL) and methylene chloride was stirred at room temperature for 2 hours. The reaction mixture was then diluted with methylene choride and washed with water, aqueous sodium bicarbonate and then brine. Evaporation of the solvent then gave 2-N-tert-butoxycarbonyl-amino-3-cyclohexylpropionic acid [1(S)-(benzoxazol-2-ylhydroxymethyl)propyl]amide (2.3 g). which was dissolved in 4N HCl in dioxane (5 mL). After stirring at room temperature for 2 h, the solvent and excess HCl were removed by evaporation and the residue was dissolved in water and freeze dried to give 2(S)-amino-3-cyclohexylpropionic acid [1(S)-(benzoxazol-2-ylhydroxy-methyl)propyl]-amide (1.5 g).

Step 2

To a solution of N-ethoxycarbonylbenzene thiamide (27 mg, 0.13 mmol) (prepared by the procedure described in Papadonpoulos, E. P., J. Org. Chem, 1976, 41, 962) and 2(S)-amino-3-cyclohexylpropionic acid [1(S)-(benzoxazol-2-ylhydroxymethyl)propyl]-amide (50 mg, 0.13 mmol) in methylene chloride was added 2-chloro-1-methylpyridinium iodide (41 mg, 0.16 mmol) and NMM (39 mg, 0.39 mmol). The reaction mixture was stirred at toom temperature overnight. After diluting the reaction mixture with water, the methylene chloride layer was separated and wahed with sat. aq. sodium bicarbonate and brine. After concentration, the crude product was purified by colum chromatography (1:1 ethyl acetate:hexane) to give ({1(S)-[1-(benzooxazol-2-ylhydroxymethyl)propylcarbamoyl]-2(S)-cyclohexylethylamino}phenyl-methylene)carbamic acid ethyl ester which was converted to the title compound as described in Example 1, Step 3 above.

### Example 3

### Synthesis of N-[1(RS)-(benzooxazol-2-ylcarbonyl)propyl]-3-cyclohexyl-2(S)-[(methanesulfonyliminopyrrolidin-1-ylmethyl)amino]propionamide

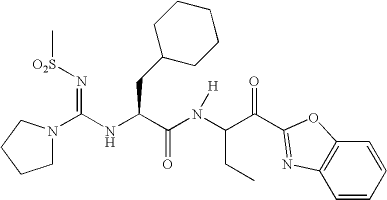 Step 1

Using the method of (H. G. McFadden, J. L. Huppatz and P. K. Halladay, Aust. J. Chem., 1993, 46, 873-886) and substituting methanesulfonamide for 2-chlorobenzenesulfonamide gave N-[bis(methylthio)methlene]methanesulfonamide.

Step 2

Cyclohexylalanine (342 mg, 2 mmol) was dissolved in water (4 mL). 1N NaOH (4 mL, 4 mmol) and N-[bis(methylthio)methlene]methanesulfonamide (400 mg, 2 mmol) were added to the reaction mixture and the reaction mixture was stirred at 70° C. for 12 h. Water (30 mL) was added to the reaction mixture and then extracted with ethyl ether. The aqueous layer was acidified with 1N HCl to pH 4. The product was extracted with ethyl acetate and the organic extracts were combined and dried over anhydrous magnesium sulfate, filtered and concentrated to afford 3-cyclohexyl-2-[(methanesulfonyliminomethylsulfanylmethyl)amino]propionic acid (664 mg) as a colorless oil. 3-Cyclohexyl-2-[(methanesulfonyliminomethylsulfanyl-methyl)amino]-propionic acid was reacted with 2(S)-amino-1-benzoxazol-2-ylbutan-1-ol to give N-[1(RS)-benzoxazol-2-ylhydroxymethyl)propyl]-3-cyclohexyl-2(R)-[(methanesulfonyl-iminomethylsulfanylmethyl)amino]propionamide.

A solution of N-[1(RS)-(benzoxazol-2-ylhydroxymethyl)propyl]-3-cyclohexyl-2(R)-[(methanesulfonylimino-methylsulfanylmethyl)amino]propionamide (254 mg, 0.5 mmol) and pyrrolidine (0.25 M) in a 5 mL microwave vial was heated in a microwave (Optimizer) at 89° C. for 1 h. The excess solvent was then removed by rotary evaporation, and the crude product was purified by silica gel chromatography (eluted with EtOAc/Hexane, 5:1) to afford N-[1(RS)-(benzoxazol-2-ylhydroxymethyl)propyl]-3-cyclohexyl-2(R)-[(methanesulfonyliminopyrrolidin-1-ylmethyl)amino]propionamide (234 mg) as white solid. N-[1(RS)-(1Benzoxazol-2-ylhydroxy-methyl)propyl]-3-cyclohexyl-2(R)-[(methanesulfonyliminopyrrolidin-1-ylmethyl)amino]-propionamide was converted to the title compound as described in Example 1, step 3 above.

The analytical data for some of the compounds of this invention are as follows:

| Cpd. # | LC/MS peaks |
|---|---|
| 1 | 536.5 (M + H), 534.3 (M − H) |
| 2 | 509.2 (M + H), 507.1 (M − H) |
| 3 | 661.6 (M + H) |
| 4 | 550(M + 1), 548(M − 1), 572(M + 23) |
| 5 | 537(M + 1), 535(M − 1), 559(M + 23) |
| 6 | 523.4(M + 1), 521.5(M − 1) |
| 9 | 502.2(M + 1), 500.4(M − 1) |
| 10 | 524.2(M + 1), 522.4(M − 1) |
| 11 | 550.2(M + 1), 548.2(M − 1) |
| 12 | 502.2 (M + 1), 500.3 (M − 1) |
| 15 | 584.4 (M + 1), 582.4 (M − 1) |
| 16 | 532.3 (M + 1), 530.3 (M − 1) |
| 17 | 520.5 (M + 1), 518.4 (M − 1) |
| 18 | 546.5 (M + 1), 544.3 (M − 1) |
| 19 | 548.1 (M + 1), 546.1 (M − 1) |
| 20 | 488.1 (M + 1), 486 (M − 1) |
| 21 | 556.5 (M + H) |
| 22 | 518.5 (M + H) |
| 23 | 581.7 (M + H) |
| 24 | 538.3 (M + H) |
| 25 | 678.4 (M + H) |
| 26 | 615.4 (M + H) |
| 27 | 551.3 (M + 1), 549.3 (M − 1) |
| 32 | 550.1(M + 1), 548.2(M − 1) |
| 33 | 551.4(M + 1), 549.3(M − 1), |
| 34 | 538.3(M + 1), 536.2(M − 1), |
| 35 | 524(M + 1), 522(M − 1), 546(M + 23) |
| 36 | 502(M + 1), 500(M − 1), 524(M + 23) |
| 37 | 502(M + 1), 500(M − 1), 524(M + 23) |
| 38 | 533(M + 1), 531(M − 1), 555(M + 23) |
| 39 | 569(M + 1), 567(M − 1), 591(M + 23) |
| 40 | 579(M + 1), 577(M − 1), 601(M + 23) |
| 41 | 597(M + 1), 595(M − 1), 619(M + 23) |
| 42 | 502(M + 1), 500(M − 1), 524(M + 23) |
| 50 | 551.6 (M + H) |

### Biological Assays

### Example 1

### Cathepsin B Assay

Solutions of test compounds in varying concentrations were prepared in 10 μL of dimethyl sulfoxide (DMSO) and then diluted into assay buffer (40 μL, comprising: N,N-bis(2-hydroxyethyl)-2-aminoethanesulfonic acid (BES), 50 mM (pH 6); polyoxyethylenesorbitan monolaurate, 0.05%; and dithiothreitol (DTT), 2.5 mM). Human cathepsin B (0.025 pMoles in 25 μL of assay buffer) was added to the dilutions. The assay solutions were mixed for 5-10 seconds on a shaker plate, covered and incubated for 30 minutes at room temperature. Z-FR-AMC (20 nMoles in 25 μL of assay buffer) was added to the assay solutions and hydrolysis was followed spectrophotometrically at (λ460 nm) for 5 minutes. Apparent inhibition constants (Kᵢ) were calculated from the enzyme progress curves using standard mathematical models.

Compounds of the invention were tested by the above-described assay and observed to exhibit cathepsin B inhibitory activity.

### Example 2

### Cathepsin K Assay

Solutions of test compounds in varying concentrations were prepared in 10 μL of dimethyl sulfoxide (DMSO) and then diluted into assay buffer (40 μL, comprising: MES, 50 mM (pH 5.5); EDTA, 2.5 mM; and DTT, 2.5 mM). Human cathepsin K (0.0906 pMoles in 25 μL of assay buffer) was added to the dilutions. The assay solutions were mixed for 5-10 seconds on a shaker plate, covered and incubated for 30 minutes at room temperature. Z-Phe-Arg-AMC (4 nMoles in 25 μL of assay buffer) was added to the assay solutions and hydrolysis was followed spectrophotometrically at (λ460 nm) for 5 minutes. Apparent inhibition constants (Kᵢ) were calculated from the enzyme progress curves using standard mathematical models.

Compounds of the invention were tested by the above-described assay and observed to exhibit cathepsin K inhibitory activity.

### Example 3

### Cathepsin L Assay

Solutions of test compounds in varying concentrations were prepared in 10 μL of dimethyl sulfoxide (DMSO) and then diluted into assay buffer (40 μL, comprising: MES, 50 mM (pH 5.5); EDTA, 2.5 mM; and DTT, 2.5 mM). Human cathepsin L (0.05 pMoles in 25 μL of assay buffer) was added to the dilutions. The assay solutions were mixed for 5-10 seconds on a shaker plate, covered and incubated for 30 minutes at room temperature. Z-Phe-Arg-AMC (1 nMoles in 25 μL of assay buffer) was added to the assay solutions and hydrolysis was followed spectrophotometrically at (λ460 nm) for 5 minutes. Apparent inhibition constants (Kᵢ) were calculated from the enzyme progress curves using standard mathematical models.

Compounds of the invention were tested by the above-described assay and observed to exhibit cathepsin L inhibitory activity.

### Example 4

### Cathepsin S Assay

Solutions of test compounds in varying concentrations were prepared in 10 μL of dimethyl sulfoxide (DMSO) and then diluted into assay buffer (40 μL, comprising: MES, 50 mM (pH 6.5); EDTA, 2.5 mM; and NaCl, 100 mM); β-mercaptoethanol, 2.5 mM; and BSA, 0.00%. Human cathepsin S (0.05 pMoles in 25 μL of assay buffer) was added to the dilutions. The assay solutions were mixed for 5-10 seconds on a shaker plate, covered and incubated for 30 minutes at room temperature. Z-Val-Val-Arg-AMC (4 nMoles in 25 μL of assay buffer containing 10% DMSO) was added to the assay solutions and hydrolysis was followed spectrophotometrically (at λ460 nm) for 5 minutes. Apparent inhibition constants (Kᵢ) were calculated from the enzyme progress curves using standard mathematical models.

Compounds of the invention were tested by the above-described assay and observed to exhibit cathepsin S inhibitory activity.

### Example 5

### Cathepsin F Assay

Solutions of test compounds in varying concentrations were prepared in 10 μL of dimethyl sulfoxide (DMSO) and then diluted into assay buffer (40 μL, comprising: MES, 50 mM (pH 6.5); EDTA, 2.5 mM; and NaCl, 100 mM); DTT, 2.5 mM; and BSA, 0.01%. Human cathepsin F (0.1 pMoles in 25 μL of assay buffer) was added to the dilutions. The assay solutions were mixed for 5-10 seconds on a shaker plate, covered and incubated for 30 minutes at room temperature. Z-Phe-Arg-AMC (2 nMoles in 25 μL of assay buffer containing 10% DMSO) was added to the assay solutions and hydrolysis was followed spectrophotometrically (at λ460 nm) for 5 minutes. Apparent inhibition constants (Kᵢ) were calculated from the enzyme progress curves using standard mathematical models.

Compounds of the invention were tested by the above-described assay and observed to exhibit cathepsin F inhibitory activity.

### Example 6

### In vitro Lip10 Accumulation Assay

During normal antigen presentation, Lip10 is proteolytically degraded to enable loading of a peptide fragment and subsequent MHC-II presentation on the surface of antigen presenting cells. The cleavage process is mediated by Cathepsin S. Thus, the Lip10 assay is an in vitro measure of a compound's ability to block cathepsin S and by extension antigen presentation. A compound that causes the accumulation of Lip10 at low concentration would be expected to block presentation of antigens.

Method:

Raji cells (4×10⁶) were cultured with 0.02% DMSO or different concentrations of Cathepsin S inhibitors in RPMI medium 1640 containing 10% (v/v) FBS, 10 mM HEPES, 2 mM L-glutamine, and 1 mM sodium pyruvate for four hours at 37° C. in 5% CO₂ humidified atmosphere. After the culture period, cells were washed with cold PBS and cells were then lysed in NP-40 lysis buffer (5 mM EDTA, 1% NP-40, 150 mM NaCl, and 50 mM Tris, pH 7.6) with protease inhibitors. Protein determinations were performed and lysate samples were boiled in reducing SDS sample buffer. Proteins were separated by electrophoresis on 12% NuPAGE® Bis-Tris gels. Proteins were then transferred to nitrocellulose membranes, and after incubation with blocking buffer (5% non-fat dry milk in PBS-Tween), the blots were incubated with the primary antibody against human CD74 invariant chain synthetic peptide (1.5 to 2 μg/ml of mouse anti-CD74 monoclonal antibody, PIN.1, Stressgen Biotechnologies). Blots were then incubated with the secondary antibody, horseradish peroxidase conjugated donkey anti-mouse IgG, at a 1:10,000 dilution. Immunoreactive proteins were detected by chemiluminescense reaction using Pierce Super Signal® West Pico chemiluminescense substrate.

### Pharmaceutical Composition Examples

The following are representative pharmaceutical formulations containing a compound of the present invention.

### Tablet Formulation

The following ingredients are mixed intimately and pressed into single scored tablets.

| Ingredient | Quantity per tablet, mg |
|---|---|
| compound of this invention | 400 |
| cornstarch | 50 |
| croscarmellose sodium | 25 |
| lactose | 120 |
| magnesium stearate | 5 |

### Capsule Formulation

The following ingredients are mixed intimately and loaded into a hard-shell gelatin capsule.

| Ingredient | Quantity per capsule, mg |
|---|---|
| compound of this invention | 200 |
| lactose, spray-dried | 148 |
| magnesium stearate | 2 |

### Suspension Formulation

The following ingredients are mixed to form a suspension for oral administration.

| Ingredient | Amount | |
|---|---|---|
| compound of this invention | 1.0 | g |
| fumaric acid | 0.5 | g |
| sodium chloride | 2.0 | g |
| methyl paraben | 0.15 | g |
| propyl paraben | 0.05 | g |
| granulated sugar | 25.5 | g |
| sorbitol (70% solution) | 12.85 | g |
| Veegum K (Vanderbilt Co.) | 1.0 | g |
| flavoring | 0.035 | mL |
| colorings | 0.5 | mg |
| distilled water | q.s. to 100 | mL |

### Injectable Formulation

The following ingredients are mixed to form an injectable formulation.

| Ingredient | Amount | |
|---|---|---|
| compound of this invention | 1.2 | g |
| sodium acetate buffer solution, | 0.4 M 2.0 | mL |
| HCl (1 N) or NaOH (1 N) | q.s. to suitable | pH |
| water (distilled, sterile) | q.s. to 20 | mL |

All of the above ingredients, except water, are combined and heated to 60-70° C. with stirring. A sufficient quantity of water at 60° C. is then added with vigorous stirring to emulsify the ingredients, and water then added q.s. to 100 g.

### Suppository Formulation

A suppository of total weight 2.5 g is prepared by mixing the compound of the invention with Witepsol® H-15 (triglycerides of saturated vegetable fatty acid; Riches-Nelson, Inc., New York), and has the following composition:

| | |
|---|---|
| compound of the invention | 500 mg |
| Witepsol ®H-15 | balance |

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled. All patents, patent applications including U.S. Provisional Application Ser. No. 60/532,234 and publications cited in this application are hereby incorporated by reference in their entirety for all purposes to the same extent as if each individual patent, patent application or publication were so individually denoted.

We claim:

1. A compound of Formula (I): 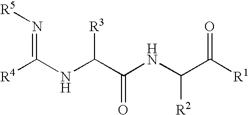
wherein:
R¹ is benzoxazol-2-yl, oxazolo-[4.5-b]-pyridin-2-yl, 2-ethyl-[1.3.4]-oxadiazol-5-yl, 2-phenyl-[1.3.4]-oxadiazol-5-yl, 3-phenyl-[1.2.4]-oxadiazol-5-yl, 3-thien-3-yl-[1.2.4]-oxadiazol-5-yl, 3-pyridin-3-yl-[1.2.4]-oxadiazol-5-yl, 3-ethyl-[1.2.4]-oxadiazol-5-yl, 5-ethyl-[1.2.4]-oxadiazol-3-yl, or 2-methoxymethyl-[1.3.4]-oxadiazol-5-yl; and
R² is ethyl or n-propyl;
R³ is cylohexylmethyl, 1-methylcyclohexylmethyl, cyclopentylmethyl, 1-methylcyclopentylmethyl, cyclopropylmethylsulfinylmethyl, cyclopropylmethylsulfonylmethyl, 2-phenylsulfanylethyl, 2-phenylsulfonylethyl, pyridin2-ylmethylsulfonylmethyl, benzylsulfinylmethyl, benzylsulfonylmethyl, 2-(difluoromethoxy)-benzylsulfonylmethyl, or 2-chlorobenzyl;
R⁴ is methyl, phenyl, 4-fluorophenyl, isopropylamine, cyclopentylamine, tetrahydropyran-4-yl, morpholin-4-yl, or pyrrolidin-1-yl;
R⁵ is methylsulfonyl, 2,2,2-trifluoroethyl, ethoxycarbonyl, or pyridin-3-ylsulfonyl; or
R⁴ and R⁵ together with the atoms to which they are attached form 1,1-dioxo-benzo[d]isothiazol-3-yl or 1,1-dioxo-1,4-dihydro-λ⁶-benzo[1.2.4]thiadiazin-3-yl; or a pharmaceutically acceptable salts thereof.

2. A compound selected from the group consisting of: 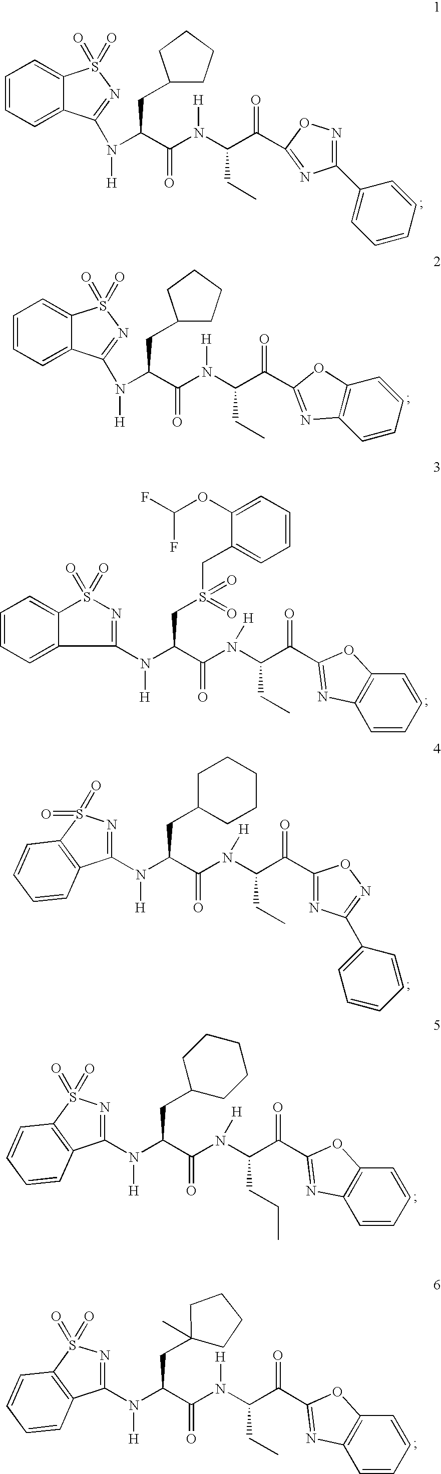 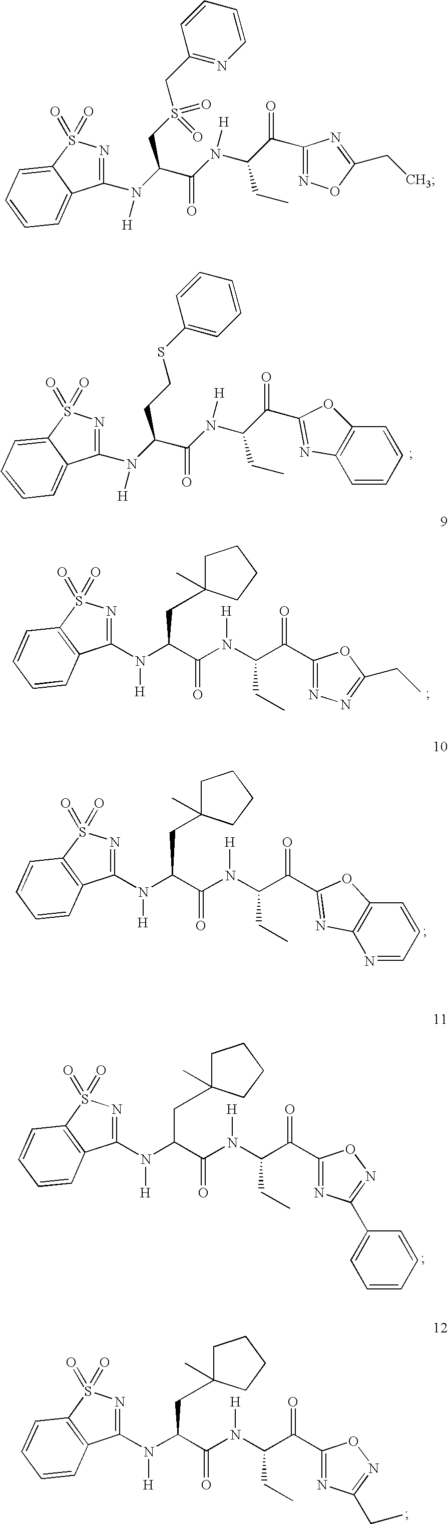 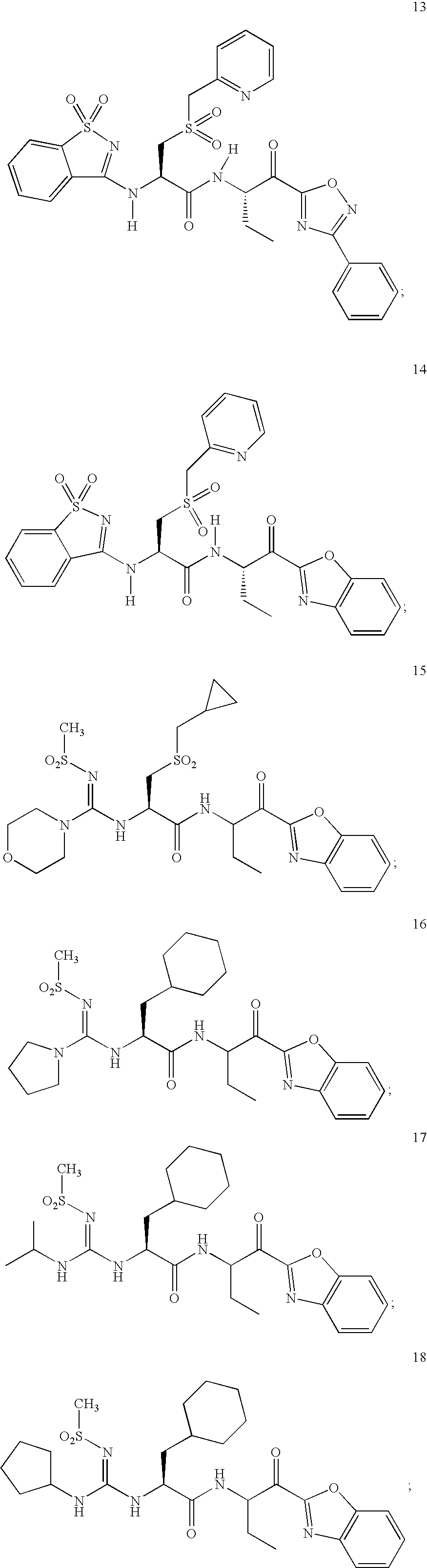 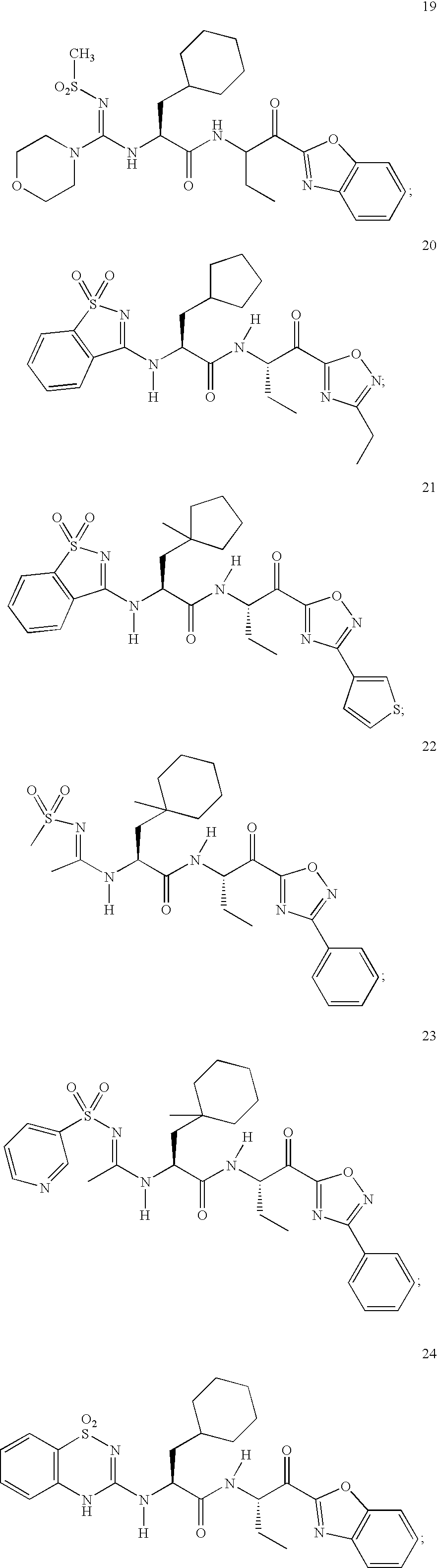 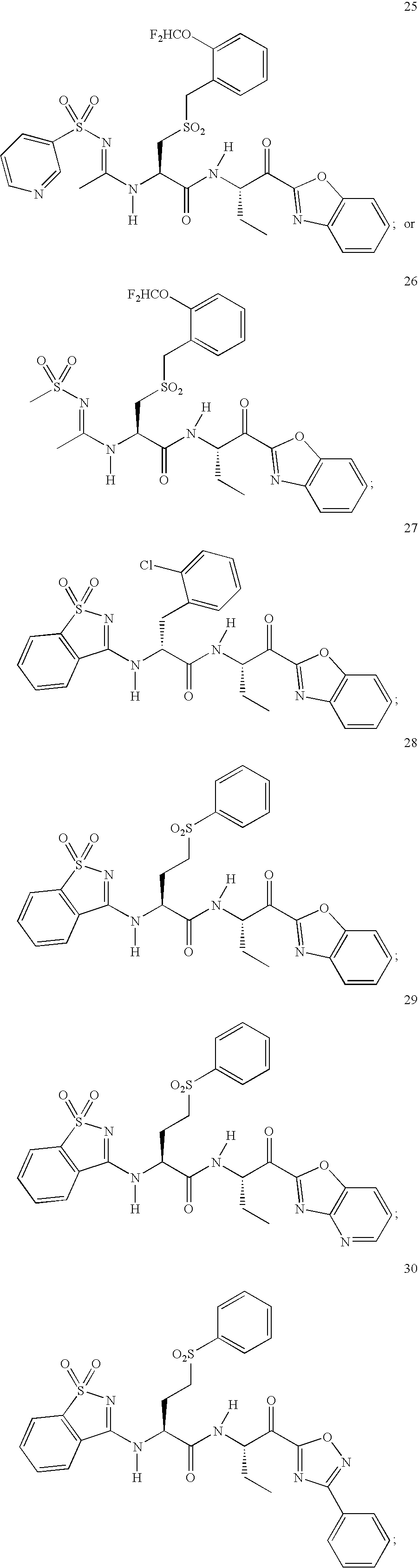 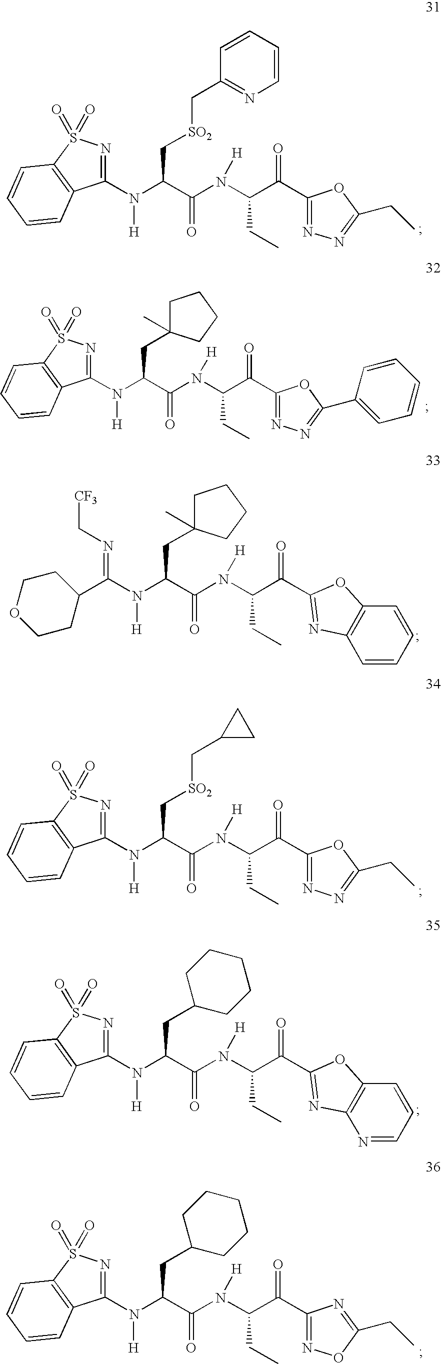 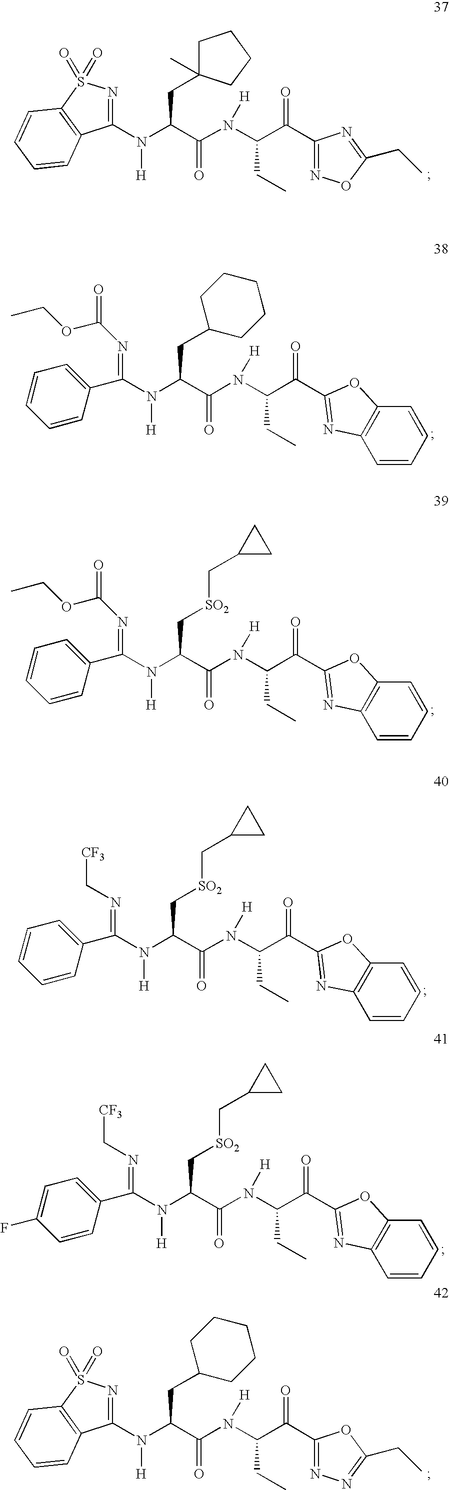 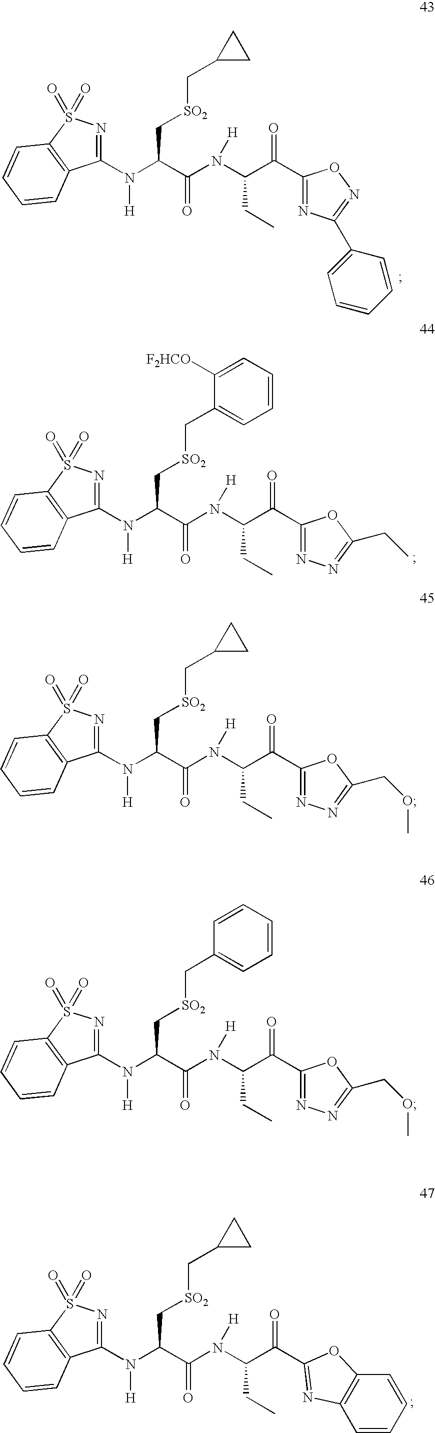 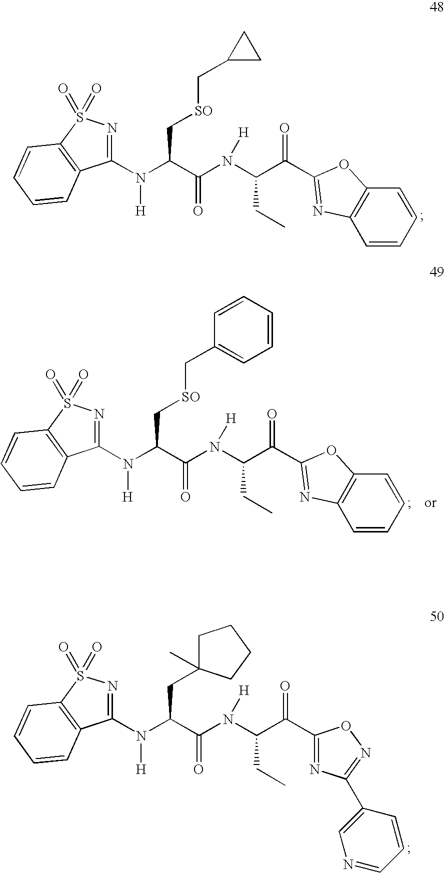
or a pharmaceutically acceptable salt thereof.

3. A compound formula:

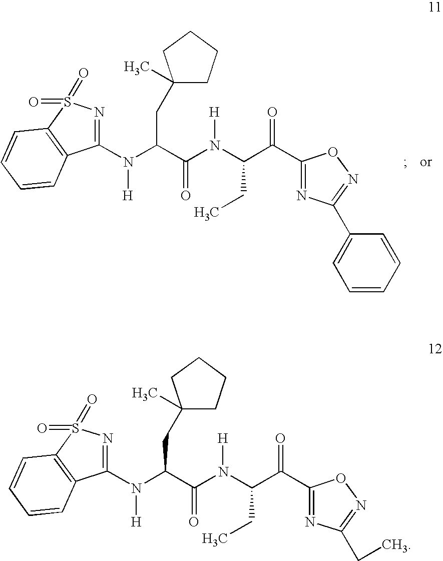

4. A pharmaceutical composition comprising a compound of any of the claims 1-3 in admixture with one or more suitable excipients.

5. A method for treating a disease in an animal mediated by cysteine proteases which method comprises administering to the animal a therapeutically effective amount of a compound of any of the claims 1-3.

6. A method of treating a patient undergoing a therapy wherein the therapy causes an immune response in the patient comprising administering to the patient a compound of any of the claims 1-3

7. The method of claim 6 wherein the therapy involves treatment with a biologic.

8. The method of claim 7 wherein the biologic is a protein.

9. The method of claim 7 wherein the biologic is an antibody.

10. The method of claim 9 wherein the biologic is Remicade®, Refacto®, Referon-A®, Factor VIII, Factor VII, Betaseron®, Epogen®, Embrel®, Interferon beta, Botox®, Fabrazyme®, Elspar®, Cerezyme®, Mybloc®, Aldurazyme®, Verluma®, Interferon alpha, Humira®, Aranesp®, Zevalin® or OKT3.

11. A method of treating immune response in an animal that is caused by administration of a biologic to the animal which method comprises administering to the animal in need of such treatment a therapeutically effective amount of a compound of any of the claims 1-3.

12. A method of conducting a clinical trial for a biologic comprising administering to an individual participating in the clinical trial a compound of any of the claims 1-3 with the biologic.

13. A method of prophylactically treating a person undergoing treatment with a biologic with a compound of any of the claims 1-3 to treat the immune response caused by the biologic in the person.

14. A method of determing the loss in the efficacy of a biologic in an animal due to the immune response caused by the biologic comprising administering the biologic to the animal in the presence and absence of a compound of any of the claims 1-3.

15. A method of improving efficacy of a biologic in an animal comprising administering the biologic to the animal with a compound of any of the claims 1-3.

16. The method of claim 5 wherein the cysteine protease is Cathepsin S.

17. The method of claim 16 wherein the disease is psoriasis or Grave's exophthalmos.

18. Use of a compound of any of the claims 1-3 for the manufacture of a medicament for combination therapy with a biologic wherein the compound treats the immune response caused by the biologic.
